(19) United States
(12) Patent Application Publication
(10) Pub. No.: US 2011/0269721 A1
(43) Pub. Date: Nov. 3, 2011

(54) METHODS OF TREATING THALASSEMIA

(75) Inventor: John D. Hood, San Diego, CA (US)

(73) Assignee: TargeGen, Inc., Sand Diego, CA (US)

(21) Appl. No.: 13/057,133

(22) PCT Filed: Aug. 3, 2009
(86) PCT No.: PCT/US09/52544

§ 371 Date: Apr. 15, 2011

Related U.S. Application Data
(62) Division of application No. 61/086,233, filed on Aug. 5, 2008.

(51) Int. Cl. A61K 31/635; A61K 31/5377; A61P 7/06; A61K 31/506

(52) U.S. Cl. 514/157; 514/275; 514/235.8; 514/252.14; 514/252.2; 514/252.19; 514/252.18

(57) ABSTRACT

Provided herein are method of treating, ameliorating, or delaying at least one symptom of a genetic blood disorder, e.g. sickle cell disorder or thalassemia, in a patient in need thereof, comprising administering a therapeutically effective amount of a Jak2 inhibitor. Also provided in part is a method of reducing an enlarged spleen in a patient suffering from thalassemia, comprising administering a therapeutically effective amount of a Jak2 inhibitor.

### RELATED APPLICATIONS

This application claims priority to U.S. Ser. No. 61/086,233 filed Aug. 5, 2008, hereby incorporated by reference in its entirety.

### BACKGROUND

Thalassemia and sickle cell anemia are genetic blood disorders that cause the formation of abnormal hemoglobin molecules, resulting in low red blood cell count. Hemoglobin consists of two different proteins, alpha and beta. If the body does not produce enough of either of these two proteins, red blood cells do not form properly and cannot carry sufficient oxygen.

Beta-thalassemia, one of the most common congenital anemias, arises from partial or complete lack of beta-globin synthesis. In both moderate and severe forms of the disease, sometimes referred to as Cooley's disease, patients may require regular blood transfusions to sustain life. Because there is no natural way for the body to eliminate iron in thalassemia patients, iron in the transfused blood cells builds up in a condition known as iron overload and becomes toxic to tissues and organs, particularly the liver and heart. As a consequence, patients are often required to undergo iron chelation therapy.

Common symptoms of thalassemia include an enlarged spleen or splenomegaly, caused by buildup of malformed red blood cells within the body. The spleen works to filter out these unhealthy cells in order to protect the body from harm, but, in a patient with thalassemia, the spleen eventually becomes enlarged and is commonly surgically removed in order to prevent a potentially fatal burst. Unfortunately, after the spleen is removed, patients are at a much greater risk for stroke and infections. Further, removal of the spleen can cause an increase in life-threatening blood clots. After a splenectomy, patients are immunocompromised, and are typically placed on lifelong prophylactic oral antibiotics.

The biological mechanism driving ineffective erythropoiesis, or ineffective production of red blood cells has not been completely understood. Accordingly, there is a need to understand these mechanisms and develop compounds useful as modulators of one or more of these processes for the treatment and/or management of genetic blood disorders such as thalassemia.

### SUMMARY

Provided herein are methods of treating and/or managing genetic blood disorders such as thalassemia and sickle cell anemia by a Jak2 inhibitor. Such Jak2 inhibitors include those compounds disclosed herein. Exemplary methods include treatment of thalassemia minor, thalassemia intermedia, and thalassemia major using Jak2 inhibitors.

In an embodiment, a method of treating, ameliorating, or delaying at least one symptom of a genetic blood disorder in a patient in need thereof is provided, comprising administering a therapeutically effective amount of a Jak2 inhibitor and/or a compound provided herein, for example, a compound represented by formula I, defined below. For example, the genetic blood disorder may be thalassemia, e.g., beta-thalassemia. In certain embodiments, methods for delaying at least one symptom of a genetic blood disorder is provided in a patient in need thereof, wherein the symptom is an enlarged spleen.

For example, a method of reducing an enlarged spleen in a patient suffering from thalassemia, is provided, comprising administering a therapeutically effective amount of a Jak2 inhibitor. A method of preventing or reducing iron overload in a patient suffering from thalassemia, is also provided, comprising administering a therapeutically effective amount of a Jak2 inhibitor.

### DESCRIPTION OF DRAWINGS

FIG. 1 depicts levels of hemolytic markers (A) bilirubin and (B) LDH(N≧6 per Genotype), (C) Epo levels in mice 2-months post-BMT and (D) Epo and Hb levels in mice up to 1 year of age. In (C), a non-parametric t-test was used for statisticalanalysis; N≧3 per genotype; p=0.0370 (*) and p=0.0008 (***), respectively, for th3/+ and th3/th3 mice compared to wildtype animals. th3/+ and th3/th3 mice are indicated respectively as +/− and −/−. Epo levels were measured in random mice up to 1 year of age or 1 year post-BMT in wildtype (squares, n=17) and th3/+(triangles, n=18) mice. Pearson's r test was used to determine the degree of linear association or the correlation coefficient between the Hb and Epo levels (wildtype, non significant, p=0.0867; th3/+, p=0.0296).

FIG. 2 depicts FACS analysis of (A) FACS analysis of CFSE-treated cells co-stained with antibodies to CD71 and Ter119. Erythroid cells from wt mice cultured in the presence of colcemid or AG490 (dashed line) showed little difference from untreated cells. Staining with 7-AAD, PI and Annexin-V excluded dead or apoptotic cells (n=4 per genotype). After 48 hours, no further cell expansion was observed, instead there is a decline in cell number, indicating that these cells did not have an intrinsic selfsustaining ability to proliferate under these tissue culture conditions; (B) FACS analysis of freshly purified erythroid cells using an antibody that recognizes the phosphorylated form of Jak2 (arrow indicating line). As a control for the specificity of the antibody, the same cells were stained with the antibody after preincubation with the competitor peptide (n=3 per genotype).

FIG. 3 depicts resulting Hb and spleen analysis after animals were administered a Jak2 inhibitor or placebo. Hb levels, spleen weight, age of the animal and days of treatment are indicated.

FIG. 4 depicts spleen size of representative animals after administration of a Jak2 inhibitor or a placebo.

### DETAILED DESCRIPTION

The present disclosure stems in part from the discovery that ineffective erythropoiesis (IE) in thalassemia is characterized by limited erythroid cell differentiation, and that thalassemic cells are associated with the expression of the cell cycle promoting gene Jak2.

Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

A “patient,” “subject” or “host” to be treated by the subject method may mean either a human or non-human animal.

The term “treating” is art-recognized and refers to curing as well as ameliorating at least one symptom of any condition or disease.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer, e.g. from 1 to 6 carbons. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Exemplary substituted alkyls are described below. Cycloalkyls may be further substituted with alkyls, alkenyls, alkoxys, alkylthios, aminoalkyls, carbonyl-substituted alkyls, —CN, and the like.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively. The term “alkylene” refers to an organic radical formed from an unsaturated aliphatic hydrocarbon; “alkenylene” denotes an acyclic carbon chain which includes a carbon-to-carbon double bond.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SOT.

The terms “amine” and “amino” are art-recognized and refer to both unsubstituted and substituted amines, e.g., a moiety that may be represented by the general formulas:

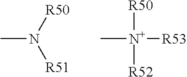

wherein R50, R51 and R52 each independently represent a hydrogen, an alkyl, an alkenyl, —(CH2)m-R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH2)m-R61. Thus, the term “alkylamine” includes an amine group, as defined above, having a substituted or unsubstituted alkyl attached thereto, i.e., at least one of R50 and R51 is an alkyl group.

The term “amido” is art recognized as an amino-substituted carbonyl and includes a moiety that may be represented by the general formula:

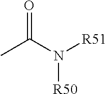

wherein R50 and R51 are as defined above. Certain embodiments of the amide in the present invention will not include imides which may be unstable.

The term “acylamino” is art-recognized and refers to a moiety that may be represented by the general formula:

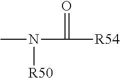

wherein R50 is as defined above, and R54 represents a hydrogen, an alkyl, an alkenyl or —(CH₂)ₘ—R61, where m and R61 are as defined above.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH2)m-R61, wherein m and R61 are defined above. Representative alkylthio groups include methylthio, ethyl thio, and the like.

The term “carbonyl” is art recognized and includes such moieties as may be represented by the general formulas:

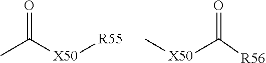

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The definition of each expression, e.g. alkyl, m, n, and the like, when it occurs more than once in any structure, is intended to be independent of its definition elsewhere in the same structure.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67^{th} Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “pharmaceutically-acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Methods

Methods of treating and/or ameloriating genetic blood disorders, such as thalassemia or sickle cell anemia, or delaying or ameliorating at least one symptom thereof, are contemplated herein, comprising administering to a patient in need thereof an effective amount of a Jak2 inhibitor, such as a small molecule Jak2 inhibitor (e.g. a Jak2 inhibitor having a molecular weight (e.g. a free base molecular weight) of about 100 g/mol to about 700 g/mol, or about 400 g/mol to about 600 g/mol. In some embodiments, the Jak2 inhibitor is represented by a compound of formula I, as provided herein. Exemplary symptoms of thalassemia include enlarged spleen and/or anemia. Symptoms may also include excessive iron absorption, and those resulting from ineffective erythropoiesis due to excessive iron absorption, including osteoporosis, e.g. secondary osteoporosis.

In an embodiment, a method is provided to ameloriate or delay an enlarged spleen in a patient suffering from thalassemia, comprising administering a pharmaceutically effective amount of a Jak2 inhibitor, e.g. a compound of formula I. For example, transfusion independent beta-thalassemia intermedia patients, if affected by splenomegaly, may develop a need for blood transfusion therapy and may eventually undergo a splenectomy. For example, patients affected by thalassmia intermedia and splenomegaly may be treated temporarily with a Jak2 inhibitor to reduce spleen size while also using blood transfusion to prevent further anemia.

In some embodiments, the spleen size of a patient suffering from thalassemia and receiving a Jak2 inhibitor may be reduced by 10%, 20%, 30%, 40%, or even 50% or more as compared to a patient with a similar spleen size suffering from thalassemia and not receiving a Jak2 inhibitor.

Methods of treating, or amelioriating or delaying at least one symptom of genetic blood disorders provided herein include methods directed to e.g., the treatment of sickle cell disease, alpha-thalassemia, delta-thalassemia, and beta-thalassemia. Contemplated treatments herein include treatment of patients suffering from thalassemia minor, thalassemia intermedia, thalassemia major (Cooley's disease), e-beta thalassemia, and sickle beta thalassemia. An exemplary method for reducing the frequency of chelation therapy in a patient, e.g., suffering from thalessemia, that includes administering a disclosed compound is provided herein.

### Compounds

Contemplated herein, for use in the provided methods are Jak2 inhibitors. In another embodiment, compounds of formula I are contemplated for use in the provided methods. Such compounds may be, e.g., Jak2 inhibitors. The compounds of formula I include those represented by:

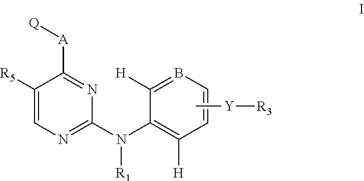

wherein A is selected from the group consisting of alkylene (e.g. C₁-C₆ alkylene) or NR₁. Q may be a monocylic or bicyclic aryl, or monocyclic or bicyclic heteroaryl, bonded to A through a ring carbon, wherein Q may be optionally substituted by 1, 2, or 3 substituents each independently selected from the group consisting of: halo, hydroxyl, cyano, amino, nitro, C₁-C₆ alkyl, C₁-C₆ alkenyl, C₁-C₆ alkynyl, C₁-C₆ alkoxy, NR₁R₁′, amido, carboxyl, alkanoyl, alkoxycarbonyl, ureido, N-alkylsulphamoyl, N-alkylcarbamoyl, carboxamide, sulphamoyl, carbamoyl, heteroaryl, heterocycle, —NR₁—C(O)—NR₁′-phenyl, SO₂NH-cycloalkyl; SO₂NH-heterocycle, SO₂H, SO₂—(C₁-C₆)alkyl, SO₂-heterocycle, or C(O)-heterocycle, wherein the heterocycle, phenyl or cycloalkyl, for each occurrence if any, may be optionally substituted by C₁-C₆ alkyl. For example, Q may be optionally substituted phenyl, naphthyl, quinoline, benzothiophene, indole, or pyridine. In a particular embodiment, Q is phenyl, optionally substituted by one N-tert-butyl sulfonamide.

R₁ and R₁′, independently, for each occurrence, can be selected from H or C₁-C₆ alkyl, e.g. may be methyl, or ethyl.

R₅ is H, cyano, or C₁-C₆ alkyl, for example, methyl, ethyl, isopropyl, n-propyl, etc. In a particular embodiment, R₅ is methyl.

B is N or CR₂, wherein R₂ is selected from the group consisting of H, halo, C₁-C₆ alkyl, C₁-C₆ alkoxy; or alkoxycarbonyl.

Y can be selected from the group consisting of: a bond, —O-alkylene; —SO₂—, SO₂—NR₁-alkylene-, —O—, alkylene, and —C(O)—, wherein R₁ is defined above. In a particular embodiment, Y is an optionally substituted methylene. In another embodiment, Y is —O—CH₂—CH₂—.

R₃ is selected from the group consisting of H, halo, hydroxyl, and R₄, wherein R₄ is a monocyclic heterocycle or heteroaryl bonded to Y through a ring carbon or heteroatom, and wherein R₄ is optionally substituted by 1, 2, or 3 substituents each independently selected from the group consisting of halo, hydroxyl, cyano, amino, nitro, C₁-C₆ alkyl, carboxyl, alkanoyl, or alkoxycarbonyl. In certain embodiments, R₄ is selected from the group consisting of pyrrolidyl, piperazinyl, morpholinyl, or piperidinyl, tetrazole, imidazole, triazole, pyrazole, or pyridinyl.

For each occurrence, if any, in formula I, C₁-C₆ alkyl, C₁-C₆ alkenyl, C₁-C₆ alkynyl, C₁-C₆ alkoxy or alkylene (e.g. C₁-C₆ alkylene) can be optionally substituted one, two, three or more times by halo, amino, hydroxyl, or cyano. Contemplated herein are also pharmaceutically acceptable salts or N-oxide thereof of compounds of Formula I.

In certain embodiments, Q can be represented by:

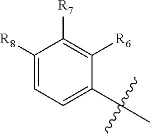

wherein R₆, R₇, and R₈ are, independently, for each occurrence, selected from the group consisting of: H, halo, hydroxyl, cyano, amino, nitro, C₁-C₆ alkyl, C₁-C₆ alkoxy, amido, carboxyl, alkanoyl, alkoxycarbonyl, N-alkylsulphamoyl, N-alkylcarbamoyl, carboxamide, sulphamoyl, carbamoyl, SO₂H, and SO₂—(C₁-C₆)alkyl.

Exemplary compounds contemplated for use in the methods provided herein include the following, or the pharmaceutically acceptable salts and/or N-oxides thereof:

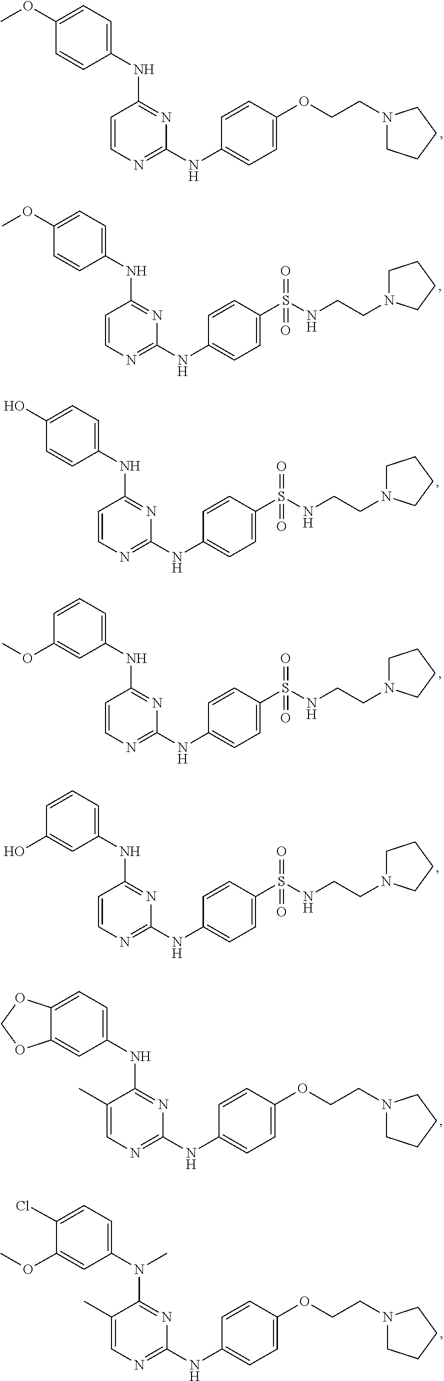

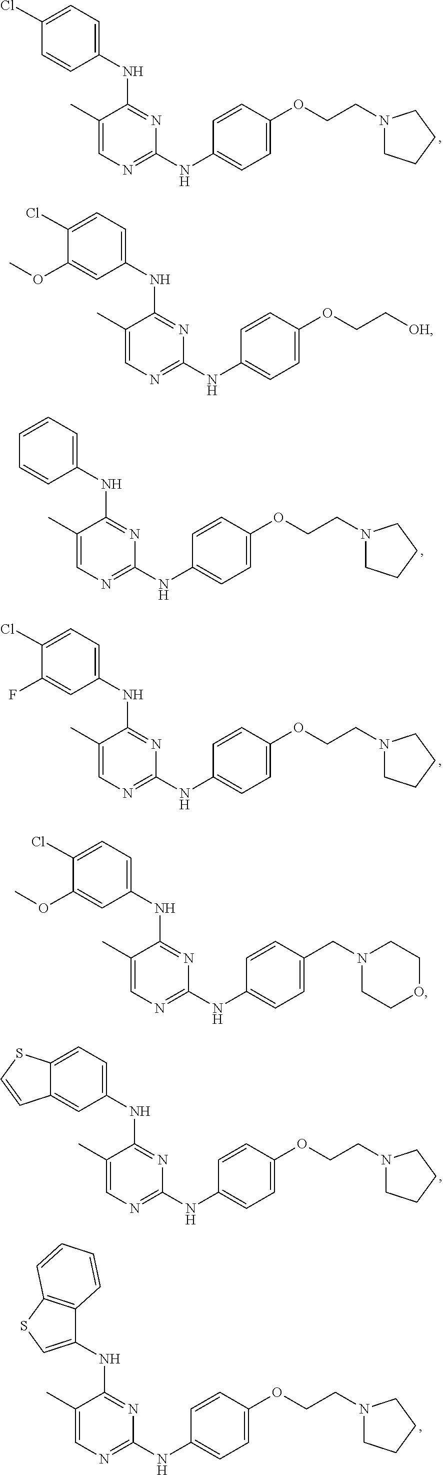

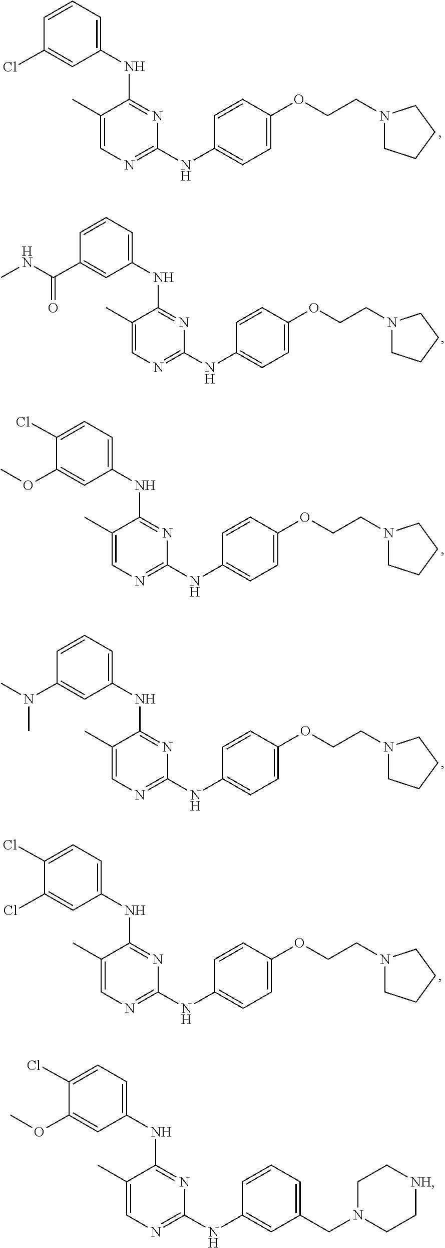

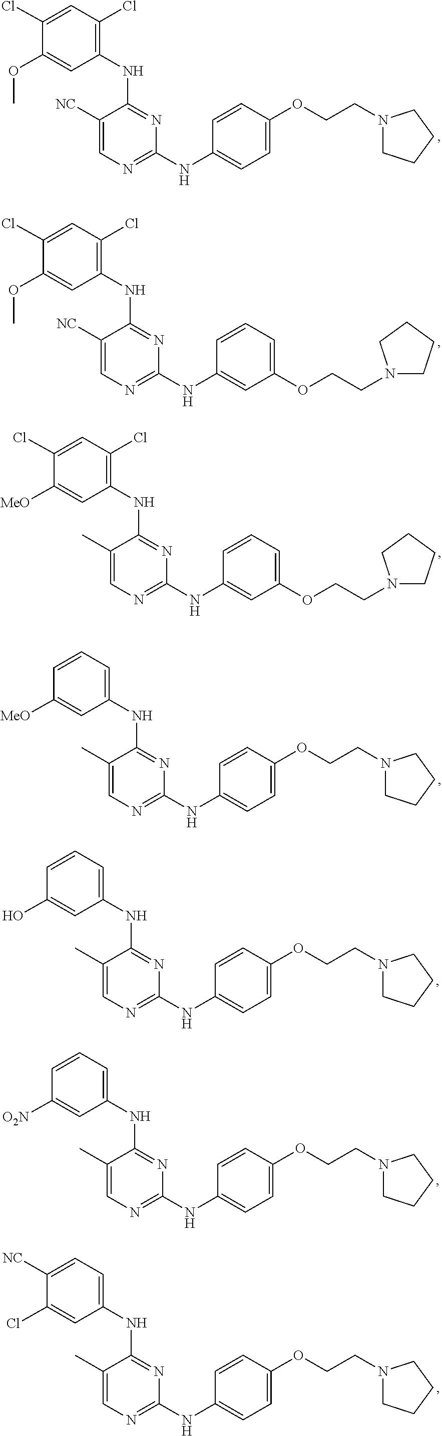

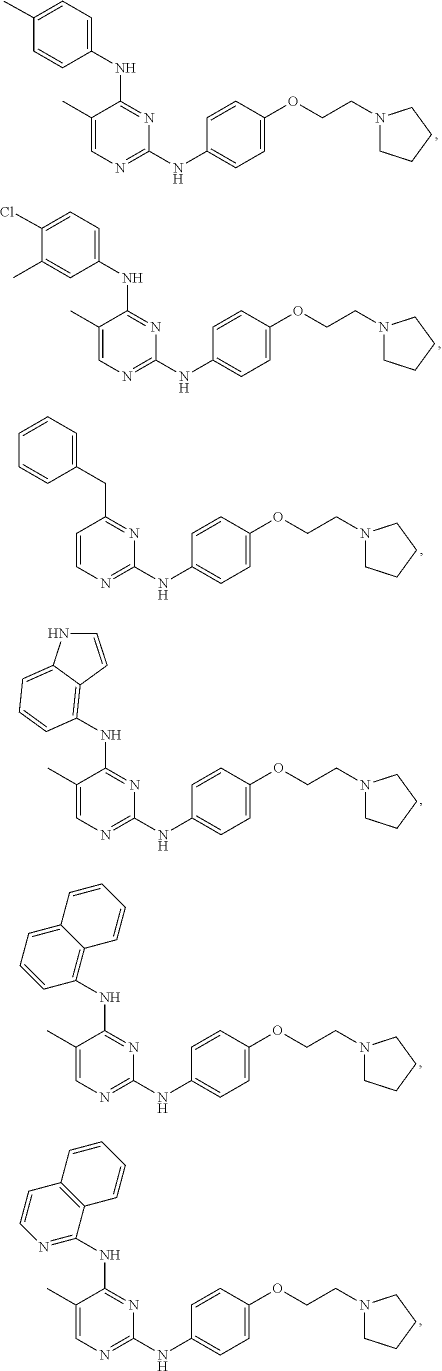

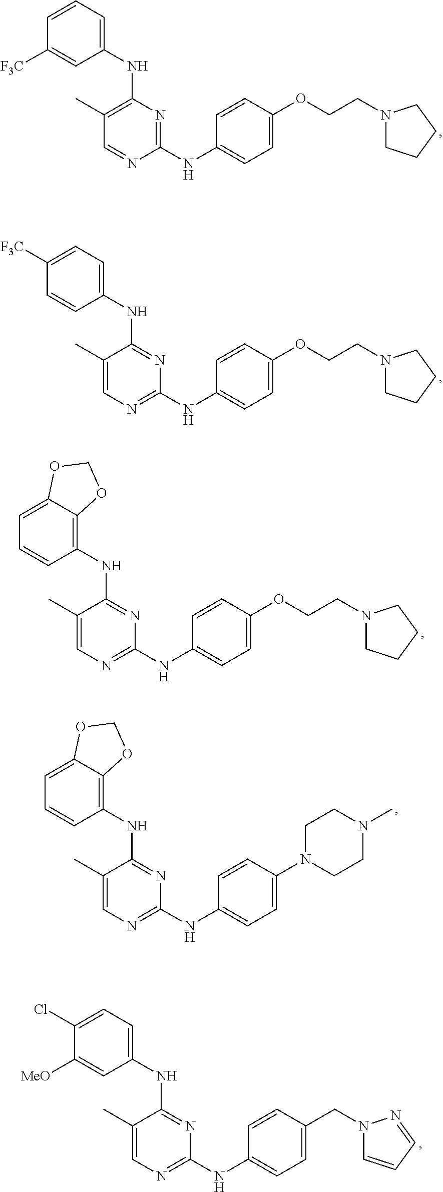

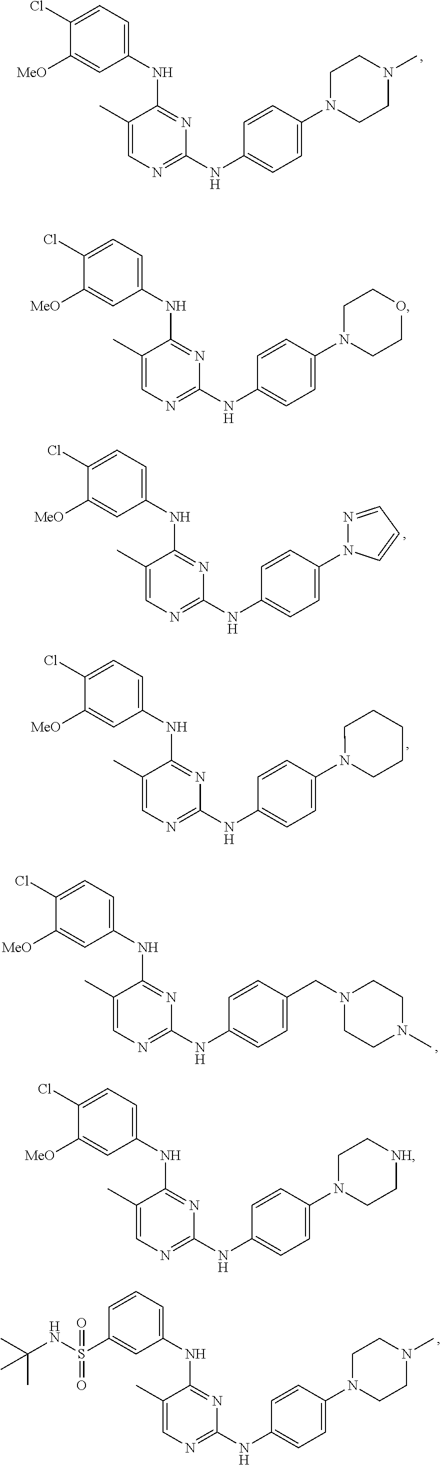

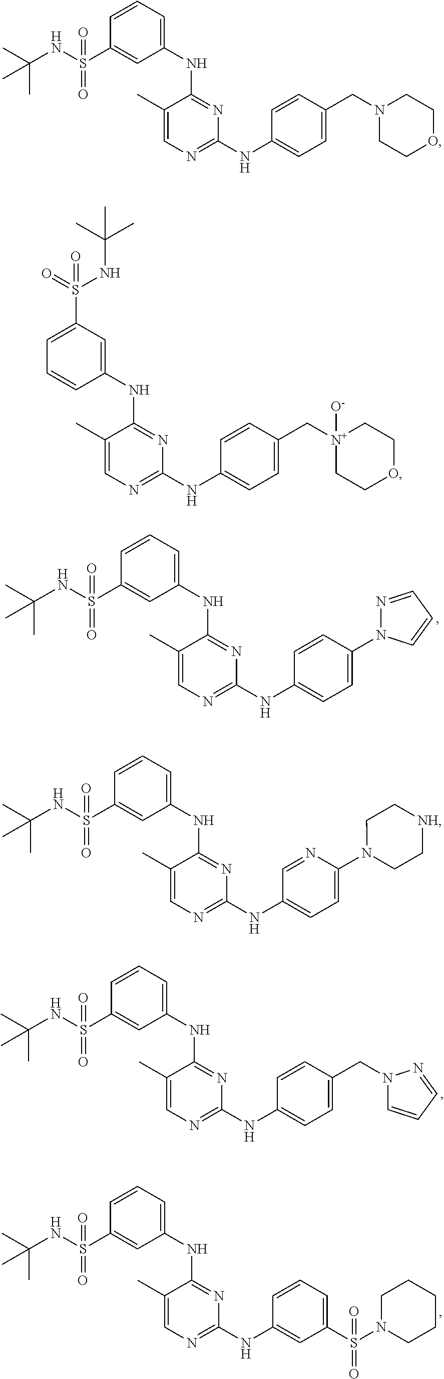

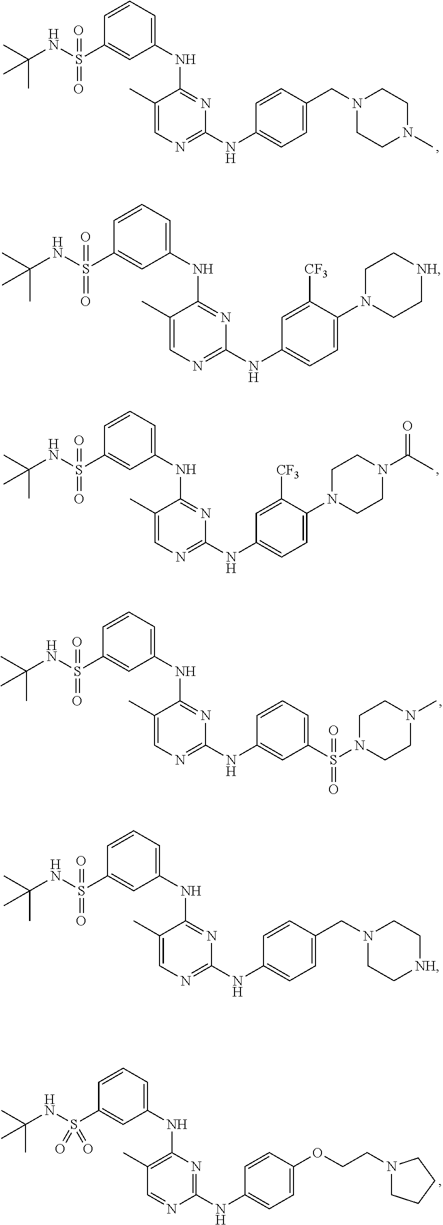

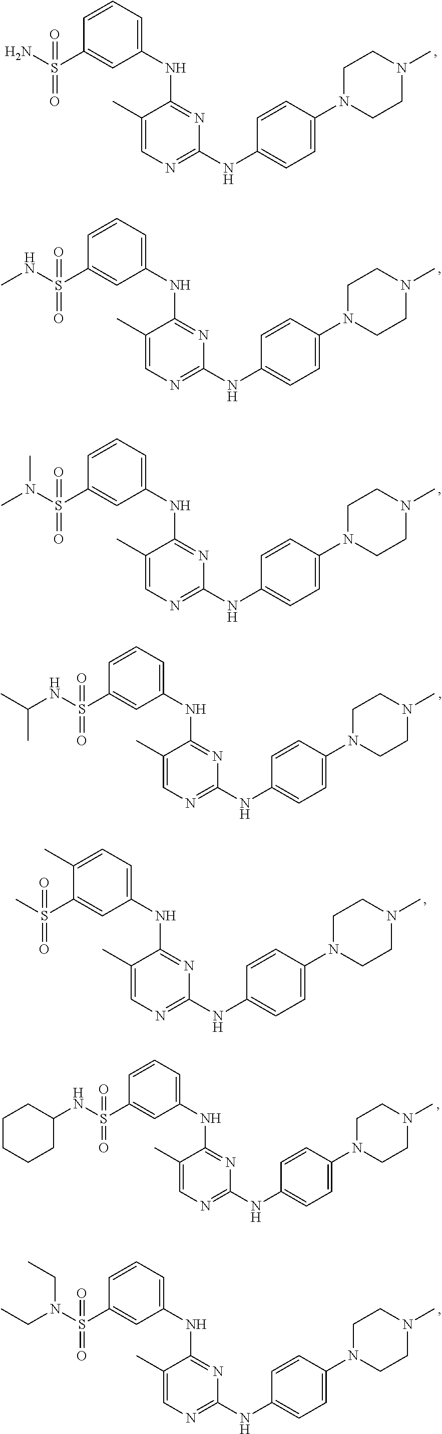

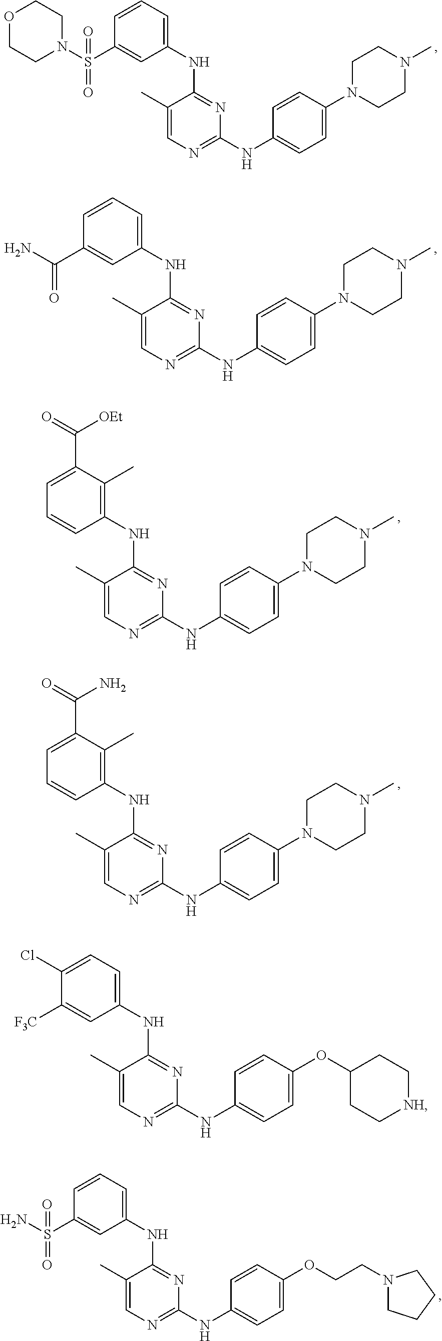

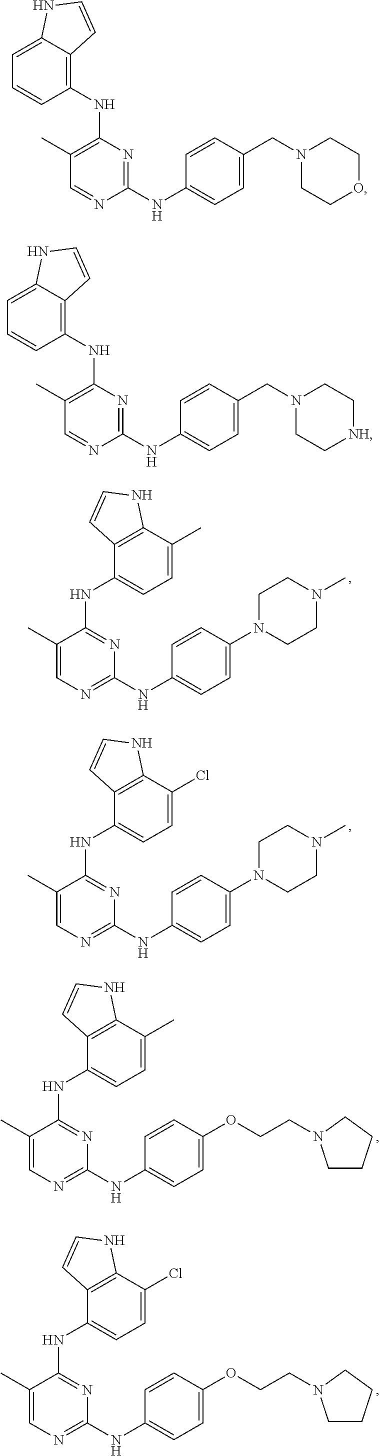

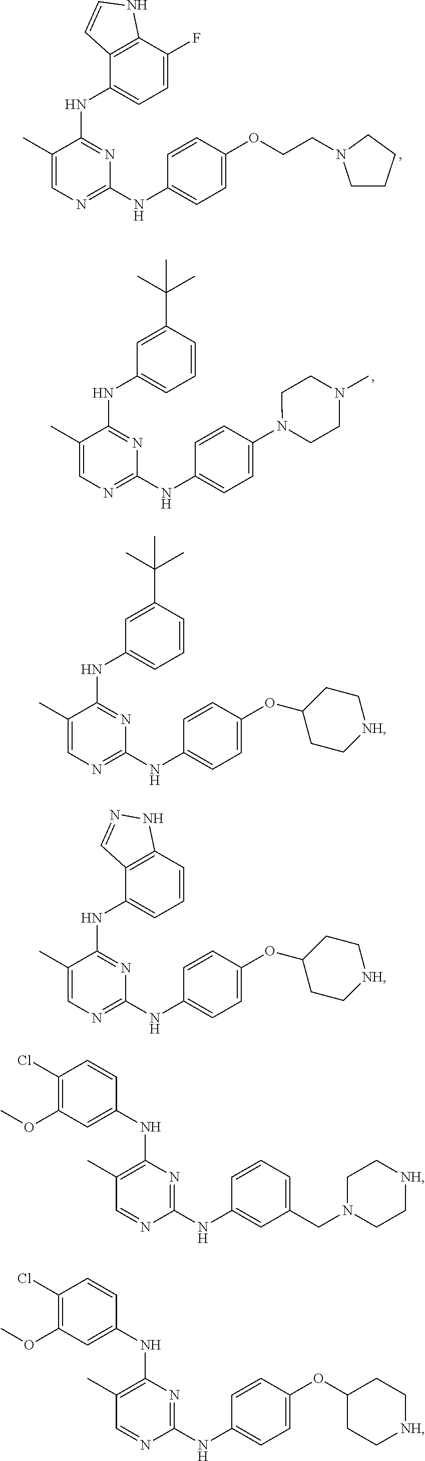

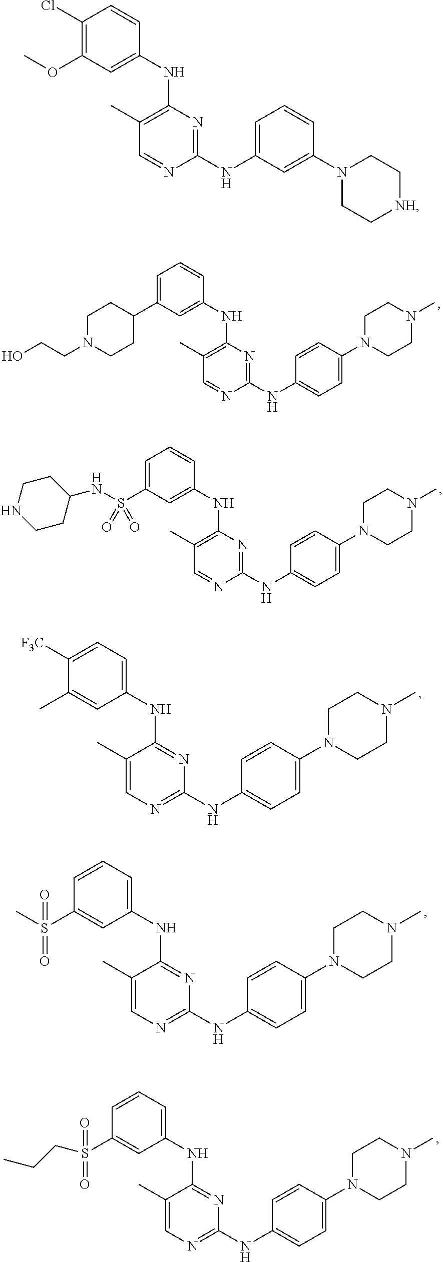

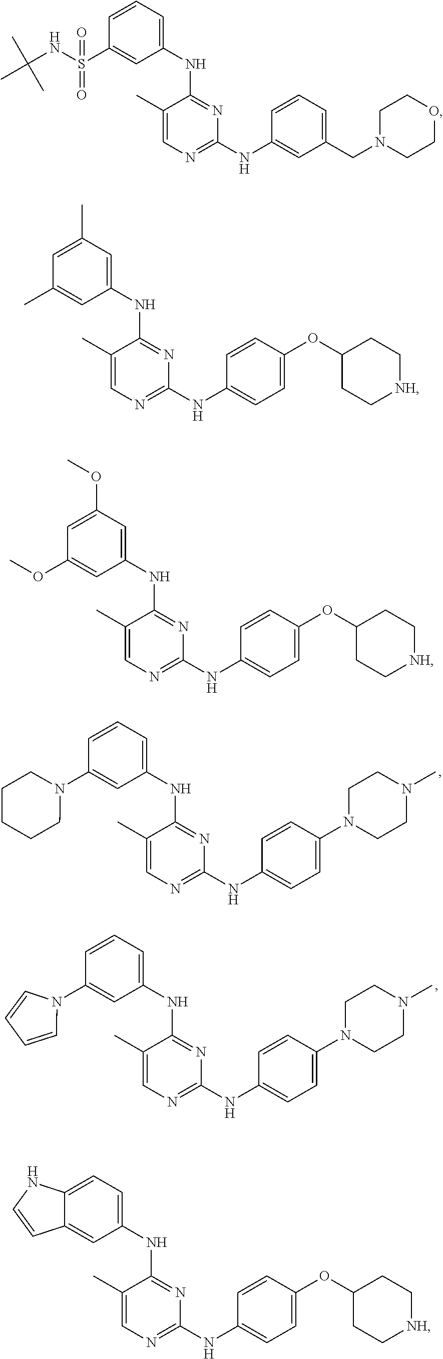

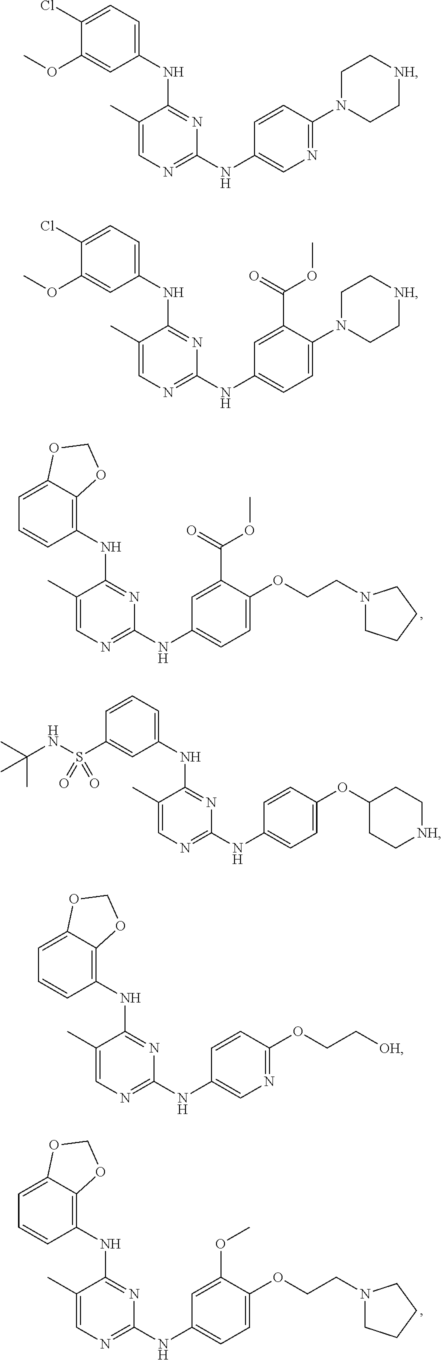

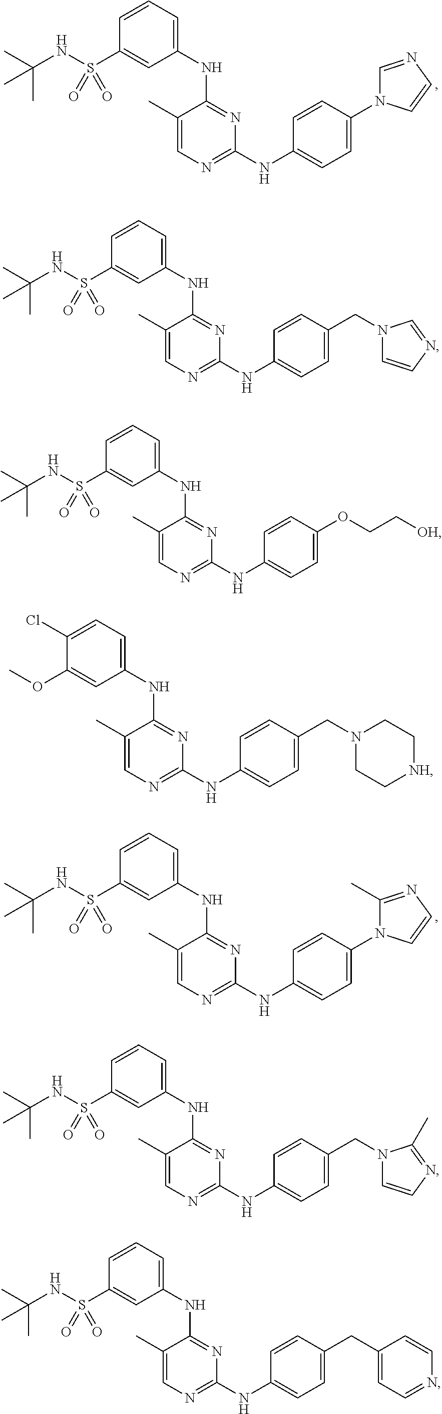

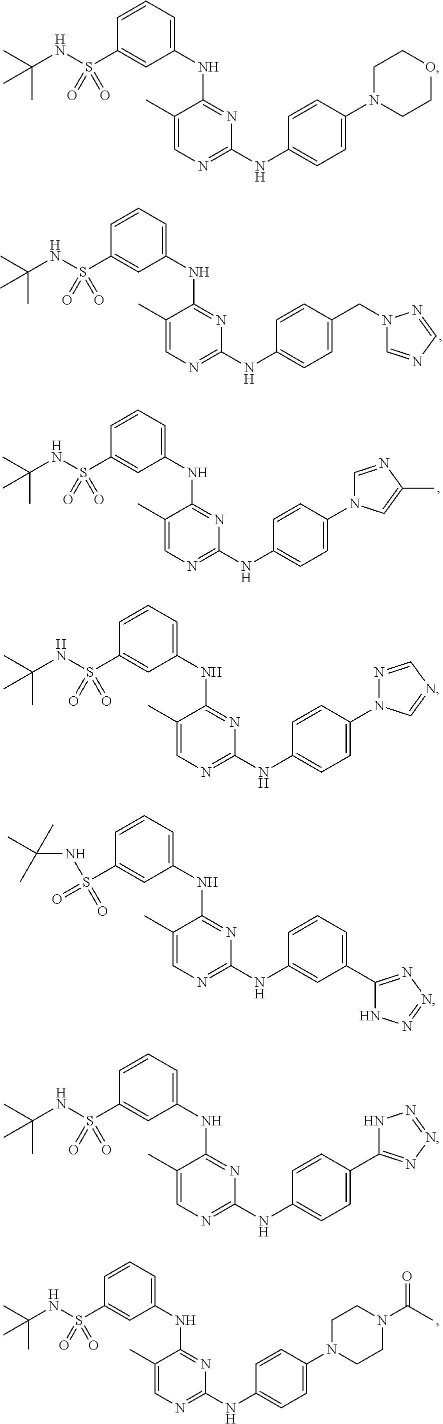

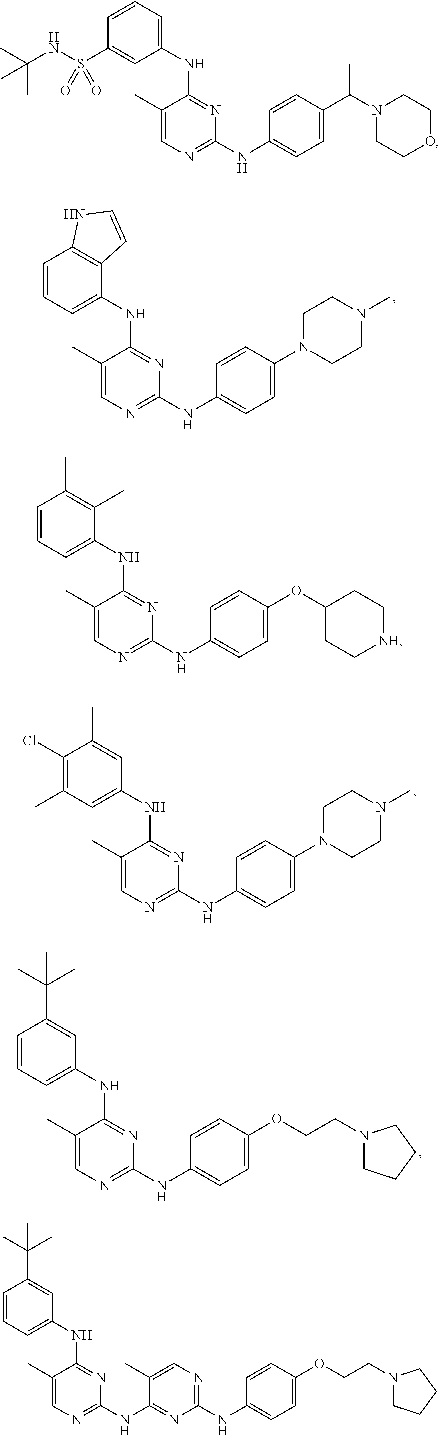

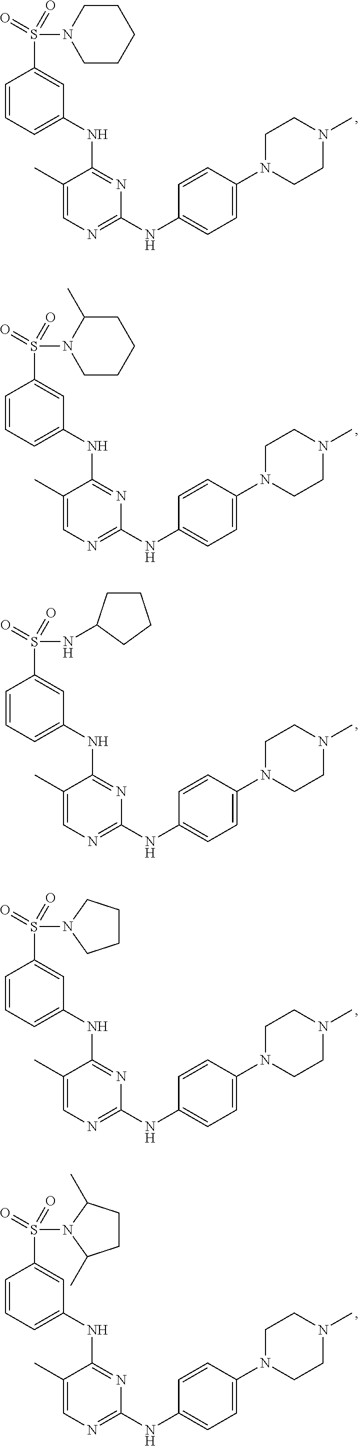

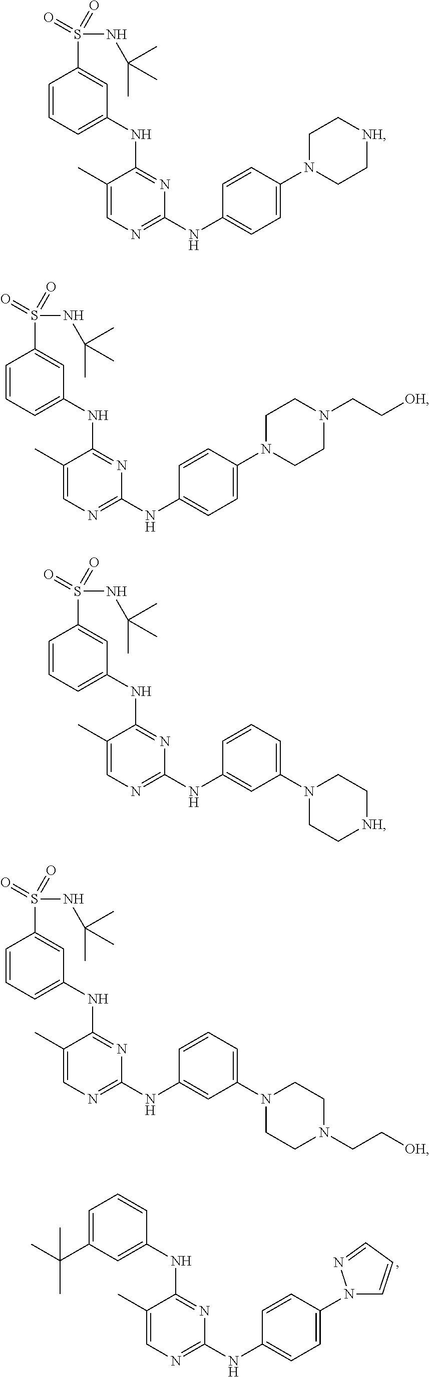

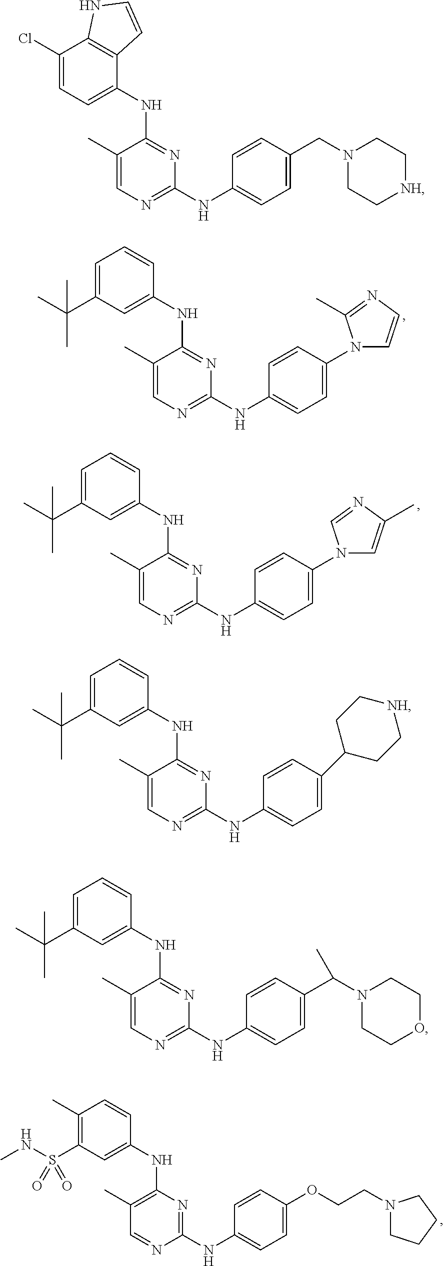

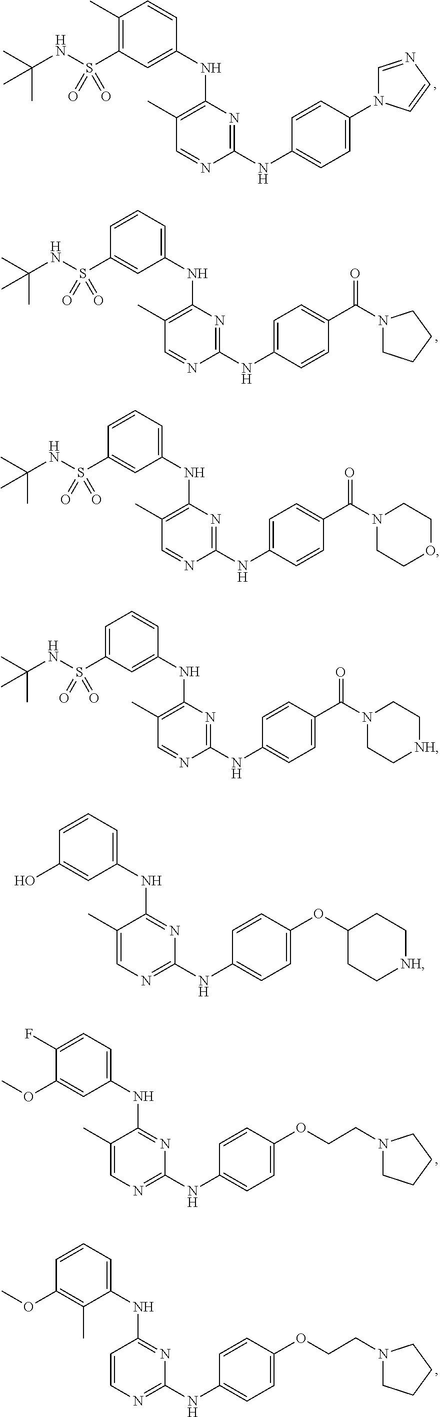

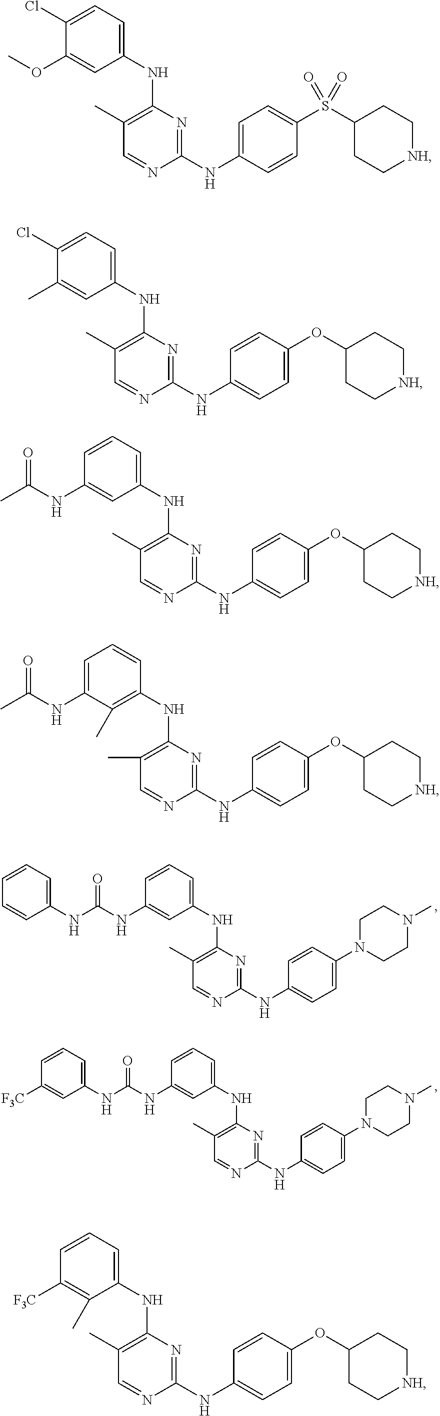

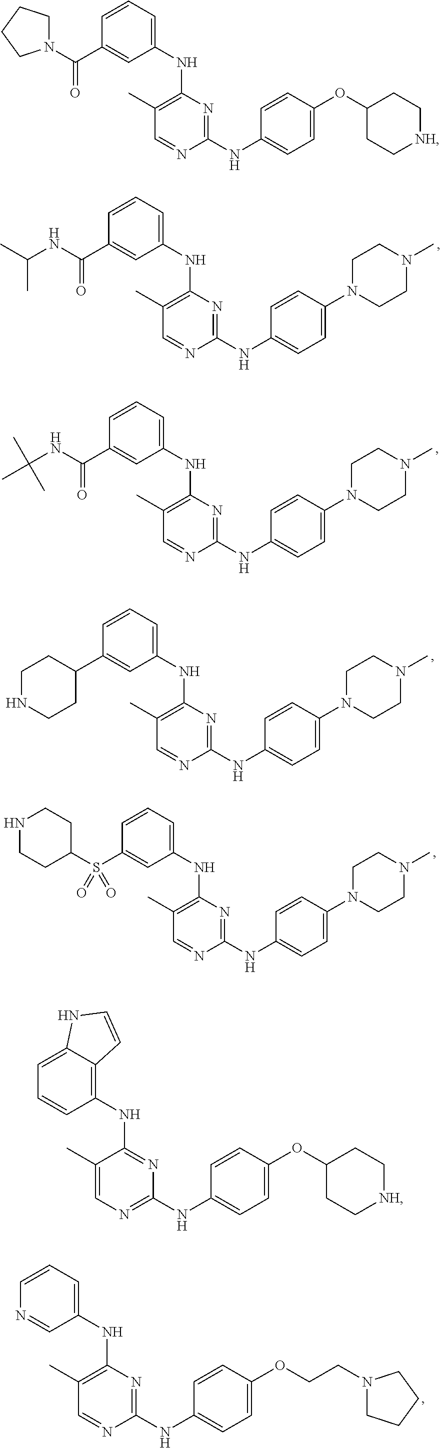

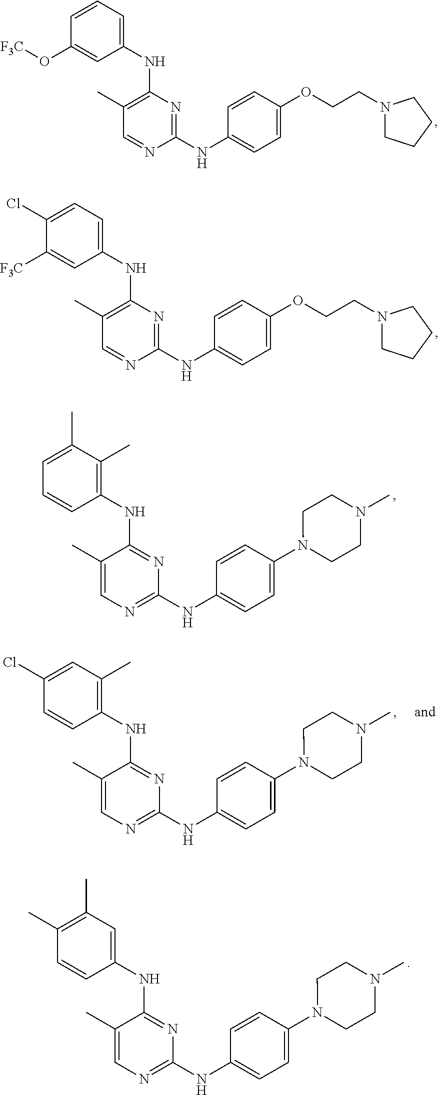

Compounds provided herein for use in the claimed methods include those compounds represented by a first moiety chemically connected to a second moiety, or a pharmaceutically acceptable salt or N-oxide thereof, wherein the first moiety is selected from the group consisting of:

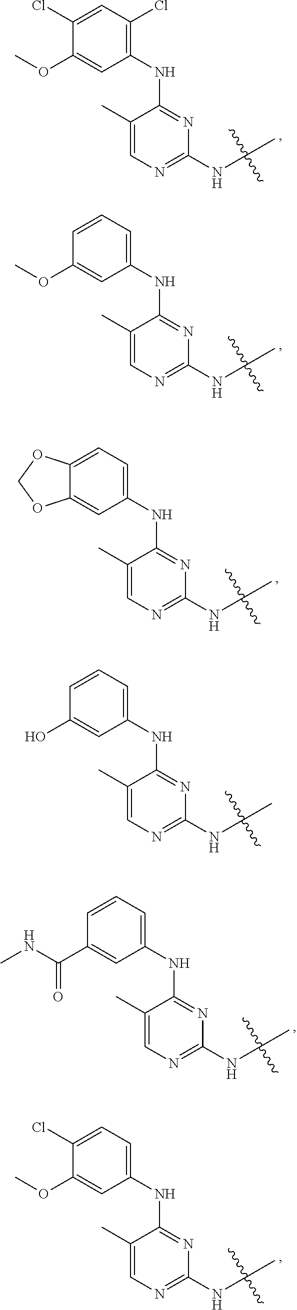

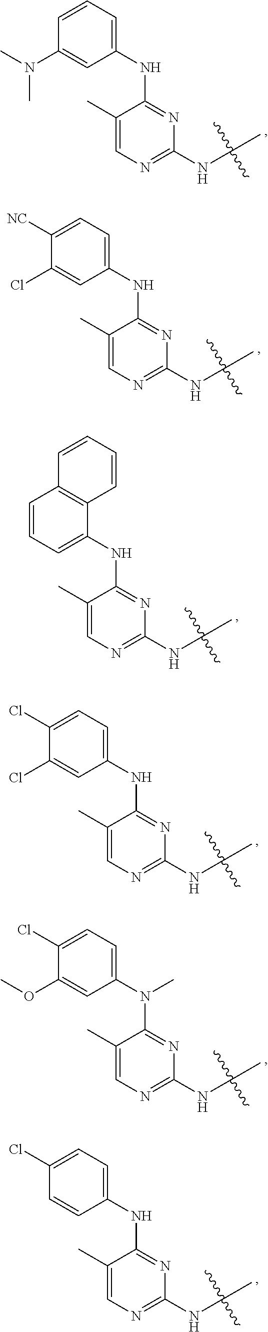

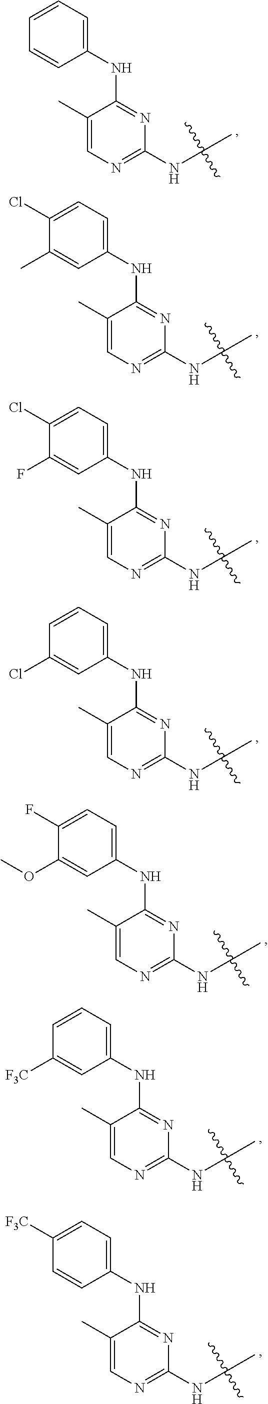

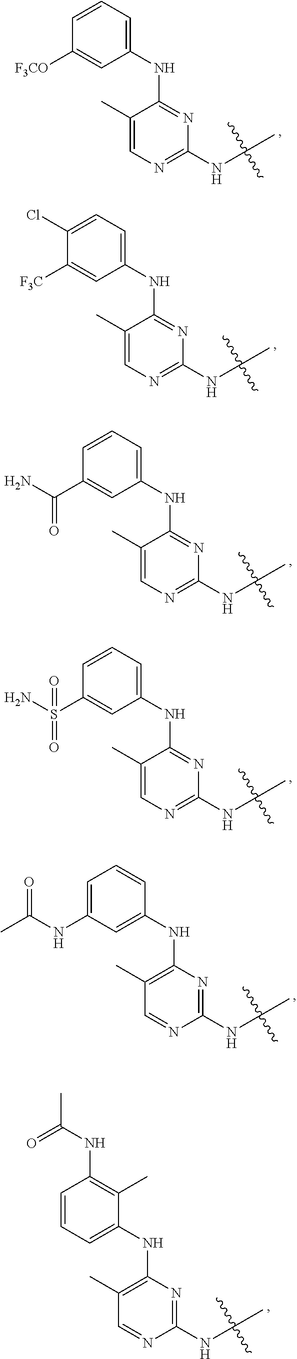

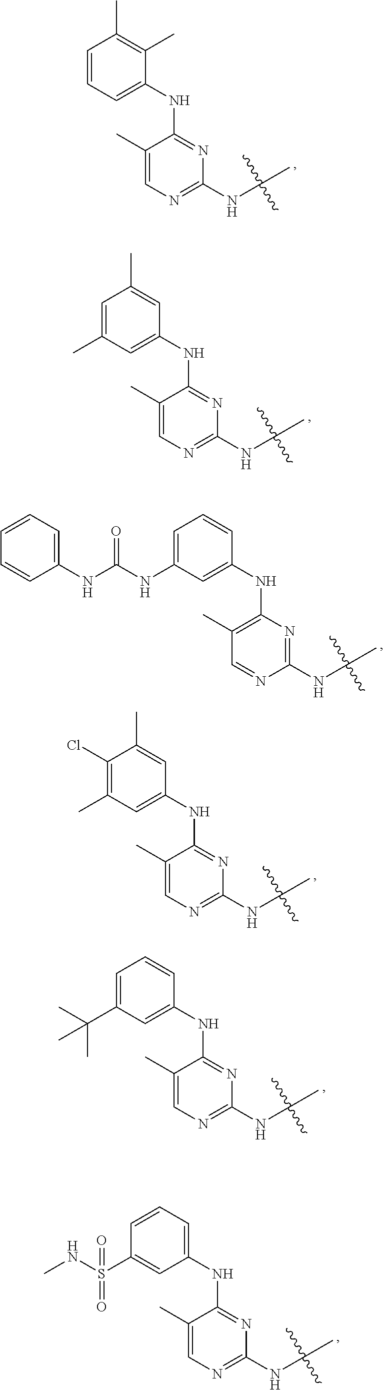

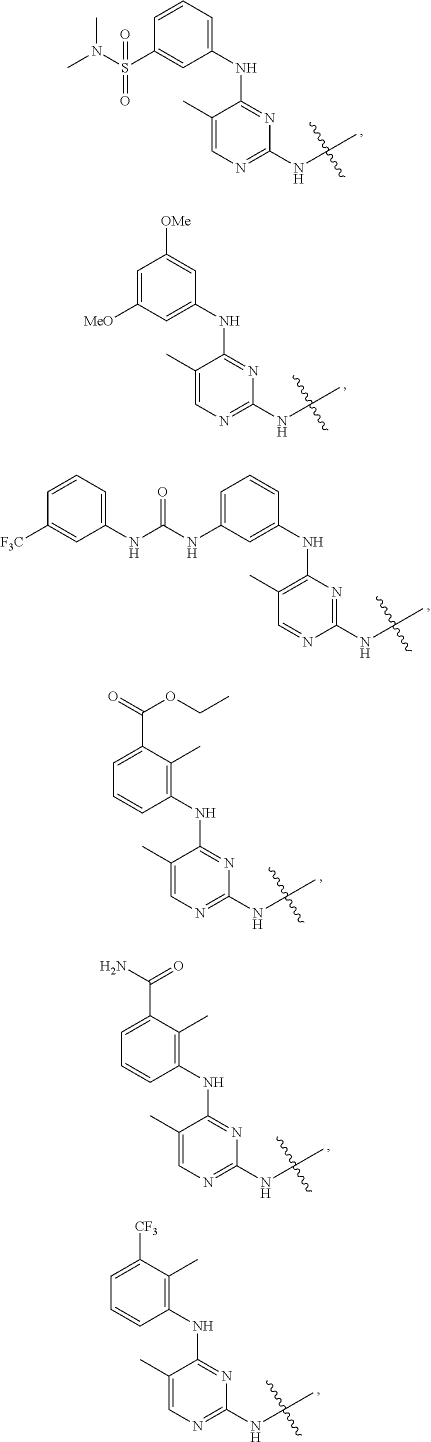

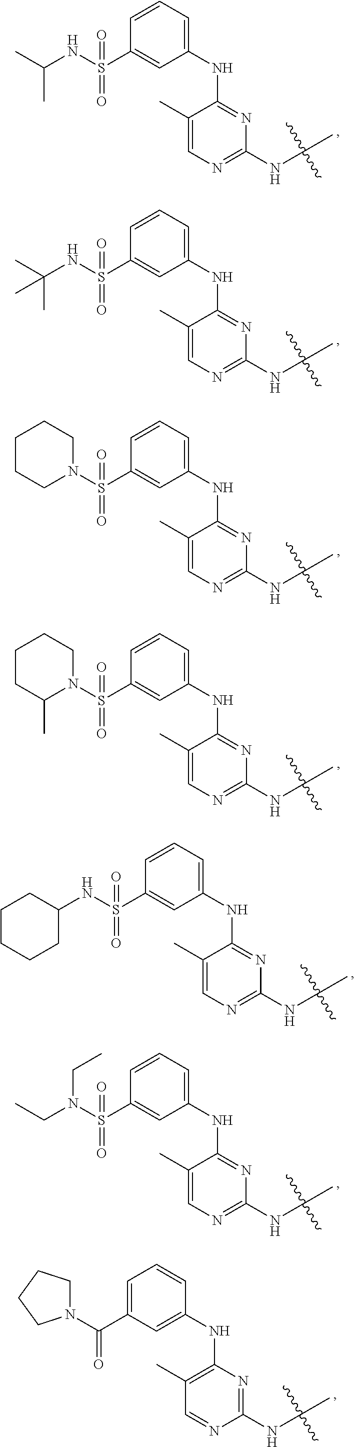

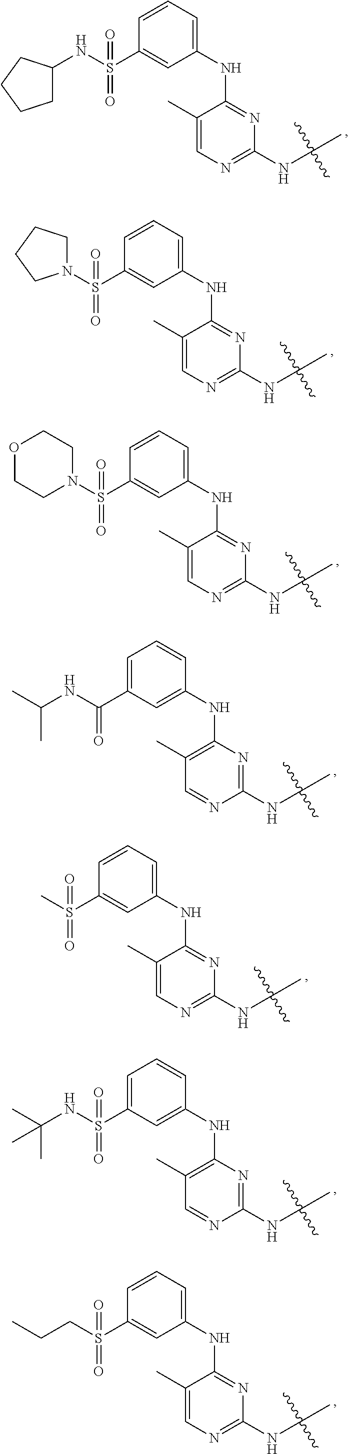

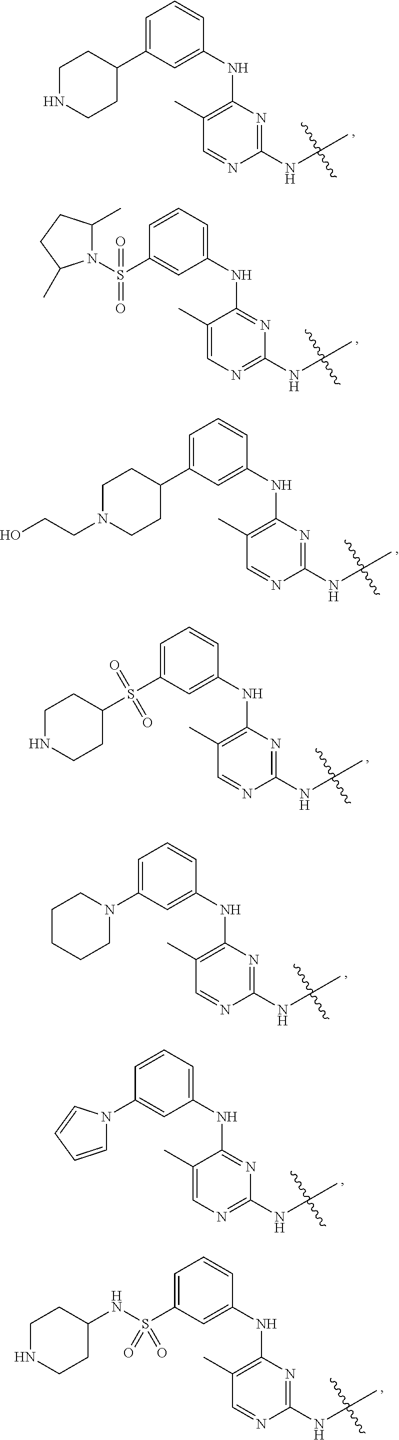

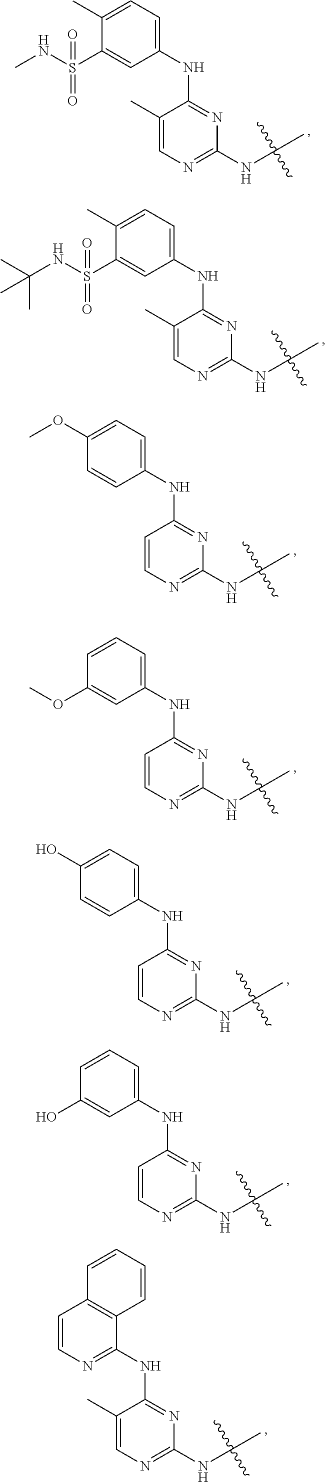

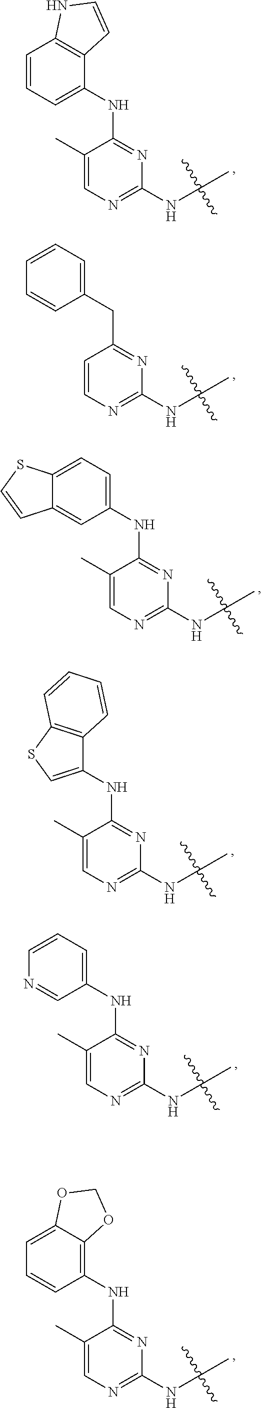

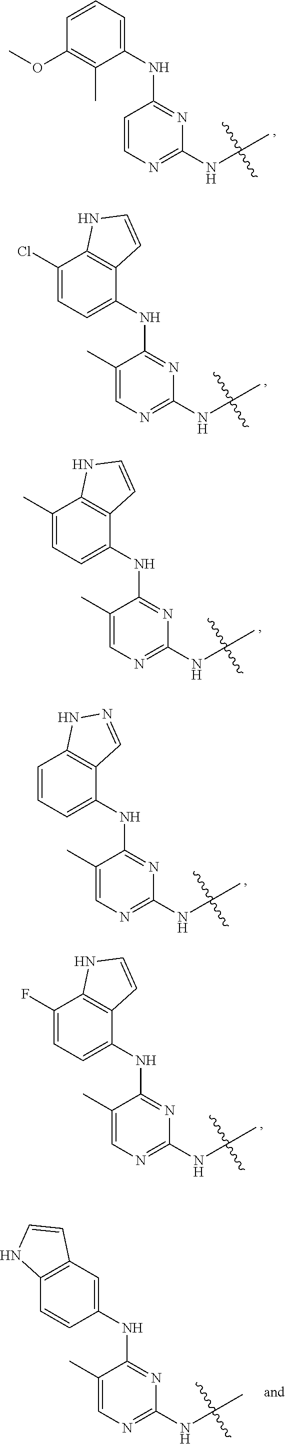

wherein the second moiety is selected from the group consisting of:

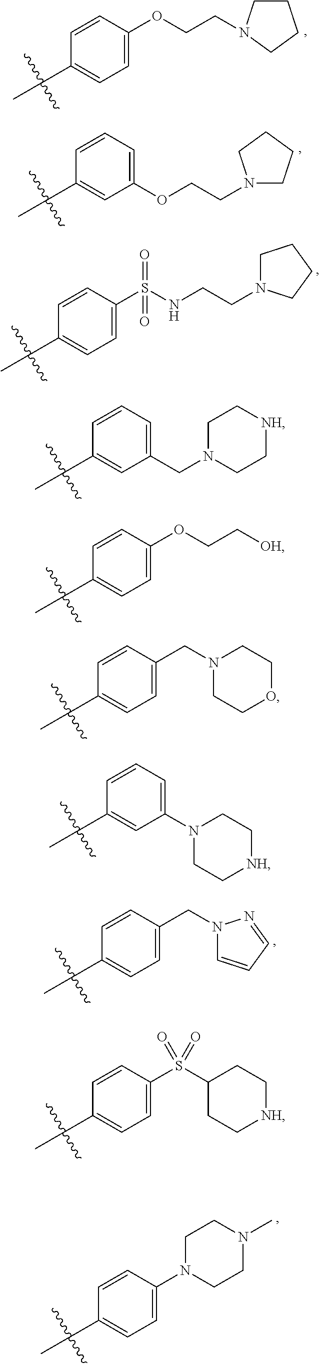

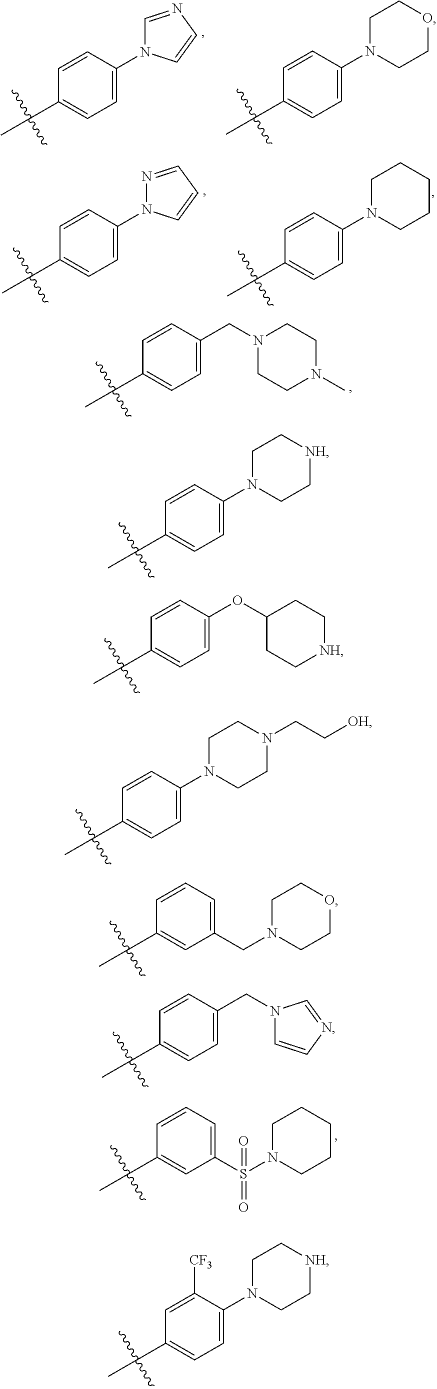

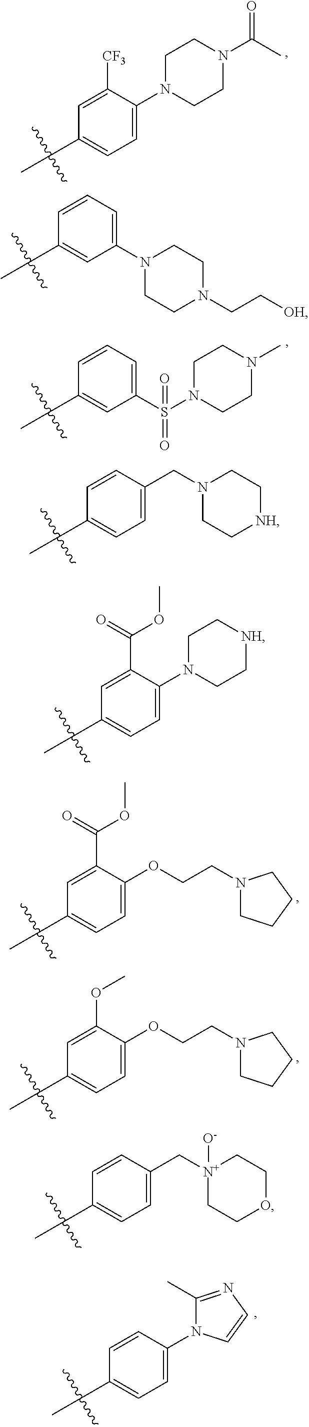

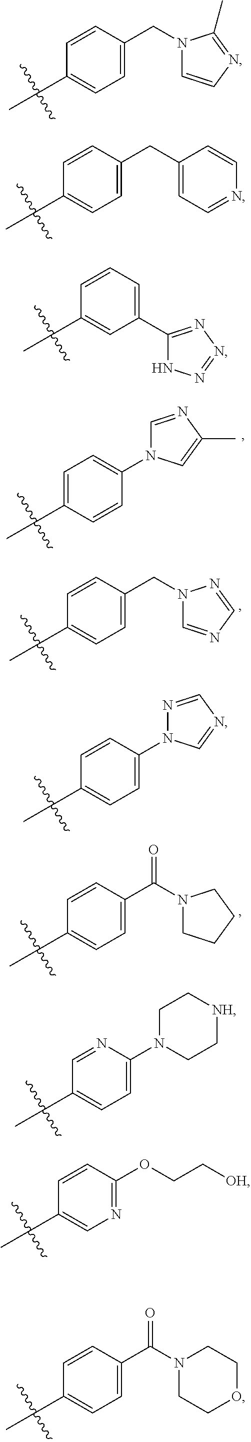

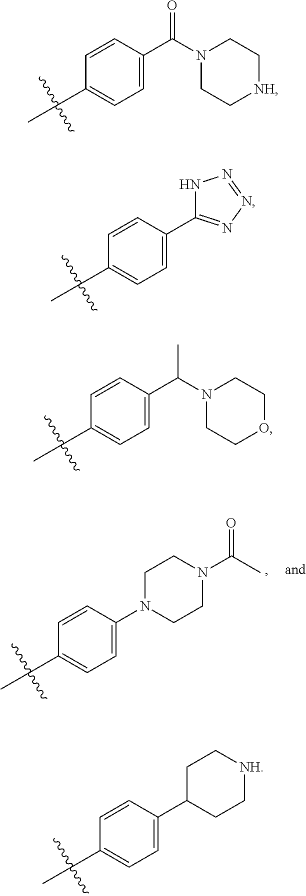

The compounds of the invention may be Jak2 inhibitors. An exemplary Jak2 inhibitor is compound A, which has the chemical structure:

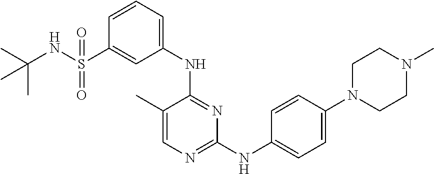

or its pharmaceutically acceptable salts thereof.

In another embodiment, an exemplary compound for use with the contemplated methods herein can be represented by:

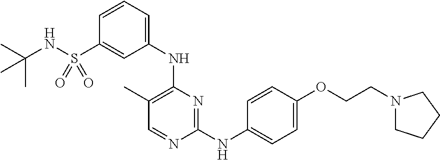

or its pharmaceutically acceptable salts thereof.

For example, IC₅₀ values for compounds can be determined using e.g. a luminescence-based kinase assay with recombinant JAK2. Such Jak2 inhibition properties are provided, for example, in U.S. Ser. No. 11/588,638, filed Oct. 25, 2006 and U.S. Ser. No. 11/796,717 filed Apr. 26, 2007, both of which are incorporated by reference in their entirety.

Compounds disclosed herein can be made using processes and procedures such as those disclosed in U.S. Ser. No. 11/588,638, filed Oct. 25, 2006 and U.S. Ser. No. 11/796,717 filed Apr. 26, 2007, both of which are incorporated by reference in their entirety.

### Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD50 and the ED50.

The data obtained from the cell culture assays and animal studies may be used in formulating a range of dosage for use in humans. The dosage of any subject composition lies preferably within a range of circulating concentrations that include the ED50 with little or no toxicity. The dosage may vary within this range depending upon the dosage form employed and the route of administration utilized. For compositions of the present invention, the therapeutically effective dose may be estimated initially from cell culture assays.

### Formulations

The compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, suppositories or administration intranasally (for example, to deliver a dosage to the brain via the nose or to deliver a dosage to the nose directly) or by inhalation (e.g. to treat a condition of the respiratory tract or to pretreat or vaccinate via the respiratory tract).

The examples which follow are intended in no way to limit the scope of this invention but are provided to illustrate how to prepare and use compounds of the present invention. Many other embodiments of this invention will be apparent to one skilled in the art.

### EXAMPLES

### General

### Mouse Models

Mouse models that mimic beta-thalassemia intermedia (th3/+) and major (th3/th3) were used. (Rivella, S. et al, A novel murine model of Cooley anemia and its rescue by lentiviral mediated human beta globin gene transfer. Blood (2003) 101: 2932-2939, hereby incorporated by reference.) In th3/+ mice both the betaminor and betamajor gens have been deleted from one chromosome. Mice completely lacking adult beta-globin (th3/th3) die late in gestation. To circumvent this problem, bone marrow transplantation is conducted wherein hematopoietic fetal liver cells (HFLCs) were harvested from th3/th3 embryos at embryonic day 14.5 (E14.5) and injected into lethally irradiated syngeneic wild-type (wt) adult recipients.

Purification of Erythroid Cells from the Spleen

Spleens from wt, th3/+ and th3/th3 mice were harvested and mechanically dissociated into single cell suspensions. Murine mononuclear cells were then isolated by centrifugation using Lympholyte-M density gradients (Cedarlane Laboratories Ltd, Westbury, N.Y.) following the manufacturer's instructions. Cells were incubated on ice for 15 minutes with a cocktail containing 10 μg each of non-erythroid FITCconjugated antibodies (GR-1, MAC1, CD4, CD8, CD11b, and CD49) (BD PharMingen). After washing, the cells were incubated for 15 minutes at 4° C. with anti-FITC microbeads (Miltenyi Biotech, Auburn, Calif.). The cell suspension was placed on a magnetic column and the eluted erythroid cells were kept for RNA extraction, protein analysis, in vitro culture with carboxy-fluorescein diacetate succinimidyl ester (CFSE; MolecularProbes, Eugene, Oreg.) staining or flow cytometric analysis.

### Primary Splenic Erythroid Cell Cultures and CFSE Staining

CFSE was added to the cells to give a final concentration of 1.25 uM. After 10 minutes at 37° C., further dye uptake was prevented by addition of 5 volumes of cold medium and incubated in ice for 5 minutes. The cells were then washed three times and seeded at 1×107 cells/ml in IMDM with 30% FBS (Hyclone, South Logan, Utah 84321), 1% deionized BSA, 100 IU/ml of penicillin, 100 ug/mL of streptomycin (Mediatech, Manassas, Va.), 70.1 mM beta-thioglycerol (mTG; Sigma-Aldrich), and 0.1 mM rHuEpo (10 U/ml; Amgen Mfg. Ltd. Thousand Oaks, Calif.). Aliquots of the cells werethen cultured in the presence and absence of 100 uM colcemid with and without AG490 (100 uM, Calbiochem-EMD Biosciences, San Diego, Calif.) or compound A, two Jak2 inhibitors.

### Phospho—Jak2 Analysis

One million cells per genotype were fixed and permeabilized (Fix and Penn Kit; Invitrogen, Grand Island, N.Y.) as per the manufacturer's instructions. Cells were incubated for 30 minutes with 0.05 ug of phospho-Jak2 polyclonal antibody (Santa Cruz Biotechnology, Santa Cruz, Calif.) or with 0.05 ug of isotype control (Santa Cruz Biotechnology). The cells were washed twice with 1% BSA-PBS and then incubated for 30 minutes at room temperature in the dark with 0.05 ug of a secondary antibody (Jackson ImmunoResearch, West Baltimore Pike, West Grove, Pa.). After washing twice, the cells were immediately analyzed using flow cytometry.

For the peptide competition assay, 0.05 ug of phospho-Jak2 polyclonal antibody were incubated for 2 h at room temperature with a 5-fold concentration of blocking peptide in Medium B of the Fix and Perm Kit. The peptide-antibody solution was then added to the fixed cells and they were incubated as above.

### Example 1

### Bilirubin and Lactic Acid Dehydrogenase (LDH) Levels in Thalassemic Mice

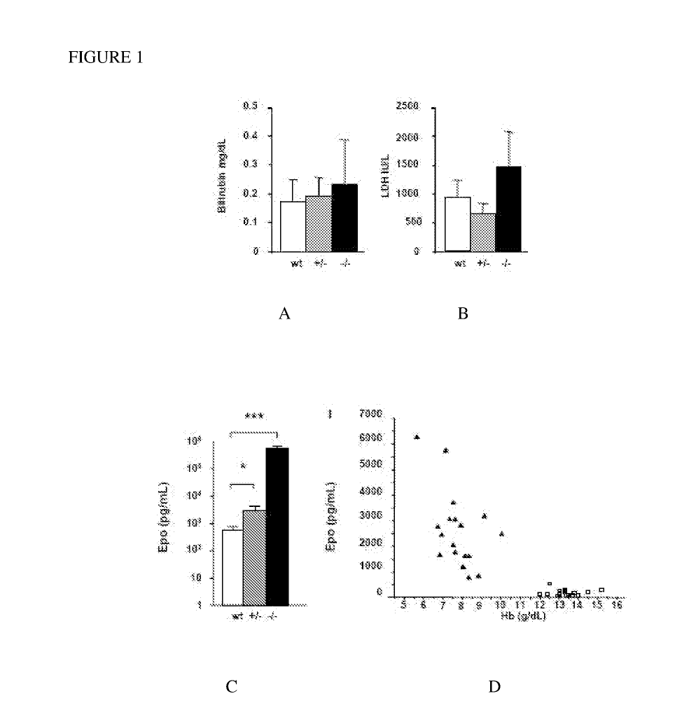

Bilirubin and lactic acid dehydrogenase (LDH) levels, which are elevated if red cells hemolyze, were unchanged or only slightly increased in thalassemic compared to normal mice (FIGS. 1A and 1B). In th3/+mice, these observations indicated that limited hemolysis was present despite erythrocyte formation. In th3/th3 erythroid cells, the average amount of alpha-globin transcript was, on average, 3 fold less than that in wt animals. The low bilirubin and LDH levels in th3/th3 mice emphasize the limited maturation of their erythroid cells, with the erythropoiesis blockade happening before the formation of fully hemoglobinized cells. Such immature morphology exhibited by thalassemic erythroid cells suggests that an altered cell cycle and limited cell differentiation may be responsible for the low levels of apoptosis and hemolysis seen in this disease compared to earlier predictions arising from ferrokinetic measurements.

### Example 2

### Jak2 Inhibition

Purified erythroid cells isolated from the spleens of normal and thalassemic mice were cultured in the presence of Epo, with and without colcemid, an anti-mitotic agent. In order to visualize cell division, the cultured erythroid cells were stained with CFSE.

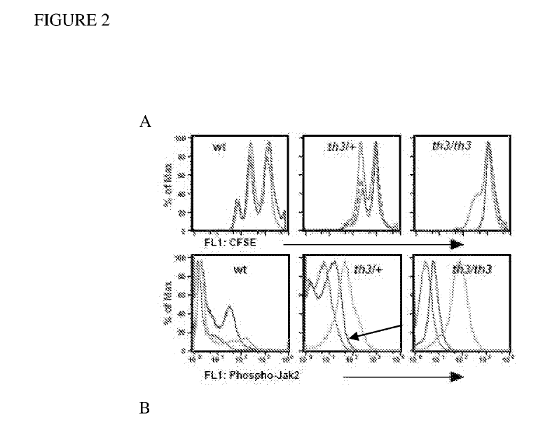

Once the dye is inside the cell, it binds to cytoskeletal proteins and is divided equally between daughter cells. Thus, it is possible to determine if cells are dividing by monitoring the reduction of CFSE fluorescence (FIG. 2A). After 48 h in culture, wt cells exhibited some differences, depending upon whether they were cultured with or without colcemid, indicating absent or limited cell proliferation (46±9% and 61±12% of the initial cell numbers, respectively, with and without colcemid; n=4). In contrast, a large proportion of the3/+ and th3/th3 cells were able toproliferate over the same time period (FIG. 2A), leading to an increase in the total cell number (th3/+, 88±18% with colcemid and 132±19% without; th3/th3, 72±25% with and 170±22% without, respectively; n=4 each genotype).

The phosphorylation of Jak2 in normal and thalassemic erythroid cells was then investigated. This analysis showed that a larger percentage of erythroid cells was positive for phospho-Jak2 (pJak2) in thalassemic compared to normal mice (FIG. 2B, n=3).

Based on these observations, the effect of Jak2 inhibitors on the erythroid cultures was investigated. AG490 and compound A, inhibitors of Jak2, had the same effect as colcemid, blocking cell proliferation (FIG. 2A, only the results for AG490 are shown). The FACS profile and the total number of cells were also similar with colcemid and the Jak2 inhibitors. Altogether, these data indicate that the increased number of proliferating cells in beta-thalassemia is associated with Jak2-mediated signaling.

### Example 3

### In Vivo Administration of Jak2

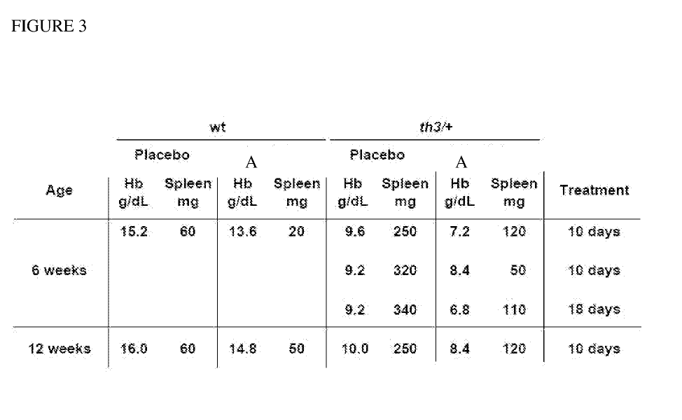

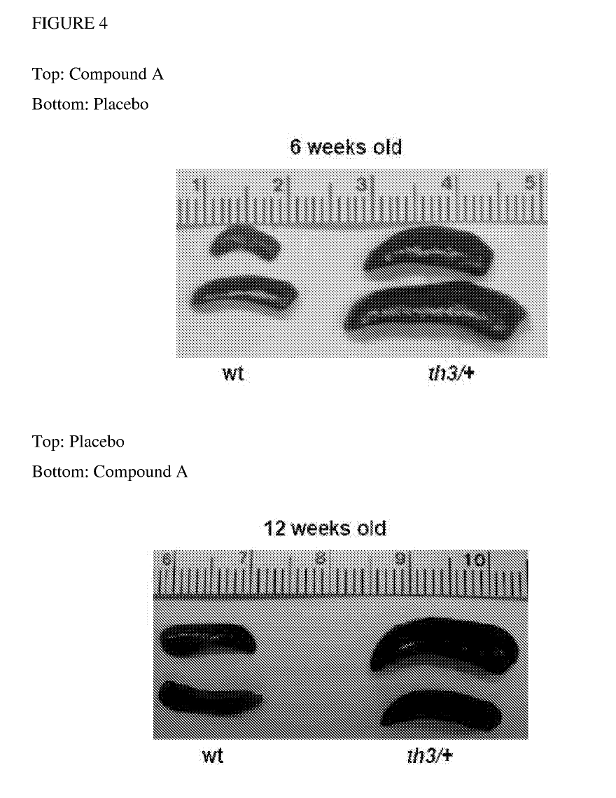

Compound A, a Jak2 inhibitor, was administered to cohorts of normal and thalassemic mice of different ages. At 10 and 18 days of treatment, the spleen size was dramatically reduced in 6- and 12-week old thalassemic mice (FIGS. 3 and 4). These changes were associated with decreasing Hb levels (FIG. 3). These observations indicate that the main role of pJak2 is to propel the erythropoietic drive. Administration of the Jak2 inhibitor also affected both erythropoiesis and the size of the spleen in young normal animals, at a time when the erythron is still expanding.

### REFERENCES

All publications and patents mentioned herein, including those items listed below, are hereby incorporated by reference in their entirety as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A method of treating, ameliorating, or delaying at least one symptom of thalassemia in a patient in need thereof, comprising administering a therapeutically effective amount of a compound represented by formula I: 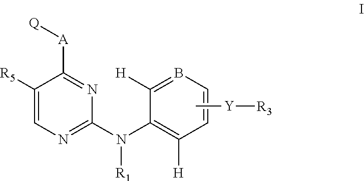
wherein
A is selected from the group consisting of alkylene or NR₁;
Q is a monocylic or bicyclic aryl, or monocyclic or bicyclic heteroaryl, bonded to A through a ring carbon,
wherein Q may be optionally substituted by 1, 2, or 3 substituents each independently selected from the group consisting of: halo, hydroxyl, cyano, amino, nitro, C₁-C₆ alkyl, C₁-C₆ alkenyl, C₁-C₆ alkynyl, C₁-C₆ alkoxy, NR₁R₁′, amido, carboxyl, alkanoyl, alkoxycarbonyl, ureido, N-alkylsulphamoyl, N-alkylcarbamoyl, carboxamide, sulphamoyl, carbamoyl, heteroaryl, heterocycle, —NR₁—C(O)—NR₁′-phenyl, SO₂NH-cycloalkyl; SO₂NH-heterocycle, SO₂H, SO₂—(C₁-C₆)alkyl, SO₂-heterocycle, or C(O)-heterocycle, wherein the heterocycle, phenyl or cycloalkyl, for each occurrence if any, may be optionally substituted by C₁-C₆ alkyl;
R₁ and R₁′, independently, for each occurrence, is selected from H or C₁-C₆ alkyl;
R₅ is H, halo, cyano, or C₁-C₆ alkyl;
B is N or CR₂;
R₂ is selected from the group consisting of H, halo, C₁-C₆ alkyl, C₁-C₆ alkoxy; or alkoxycarbonyl;
Y is selected from the group consisting of: a bond, —O-alkylene; —SO₂—, SO₂—NR₁-alkylene-, —O—, alkylene, and —C(O)—;
R₃ is selected from the group consisting of H, halo, hydroxyl, and R₄, wherein R₄ is a monocyclic heterocycle or heteroaryl bonded to Y through a ring carbon or heteroatom, and wherein R₄ is optionally substituted by 1, 2, or 3 substituents each independently selected from the group consisting of halo, hydroxyl, cyano, amino, nitro, C₁-C₆ alkyl, carboxyl, alkanoyl, or alkoxycarbonyl;
wherein for each occurrence, if any, C₁-C₆ alkyl, C₁-C₆ alkenyl, C₁-C₆ alkynyl, C₁-C₆ alkoxy or alkylene can be optionally substituted by halo, amino, hydroxyl, or cyano;
or a pharmaceutically acceptable salt or N-oxide thereof.

2. The method of claim 1, wherein Q is selected from the group consisting of phenyl, naphthyl, quinoline, benzothiophene, indole, or pyridine.

3. The method of claim 1, wherein R₅ is C₁-C₆ alkyl.

4. The method of claim 1, wherein R₅ is methyl.

5. The method of claim 1, wherein Y is methylene.

6. The method of claim 1, wherein Y is —O—CH₂—CH₂—.

7. The method of claim 1, wherein R₄ is selected from the group consisting of pyrrolidyl, piperazinyl, morpholinyl, or piperidinyl.

8. The method of claim 1, wherein R₄ is selected from the group consisting of tetrazole, imidazole, triazole, pyrazole, or pyridinyl.

9. The method of claim 7, wherein R₄ is substituted with a methyl.

10. The method of claim 1, wherein Q is phenyl.

11. The method of any claim 1, wherein Q is substituted by N-tert-butyl sulfonamide.

12. The method of claim 1, wherein Q is represented by: 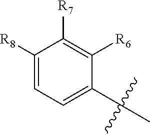
wherein R₆, R₇, and R₈ are, independently, for each occurrence, selected from the group consisting of: H, halo, hydroxyl, cyano, amino, nitro, C₁-C₆ alkyl, C₁-C₆ alkoxy, amido, carboxyl, alkanoyl, alkoxycarbonyl, N-alkylsulphamoyl, N-alkylcarbamoyl, carboxamide, sulphamoyl, carbamoyl, SO₂H, and SO₂—(C₁-C₆)alkyl.

13. The method of claim 1, wherein the compound is selected from the group consisting of:

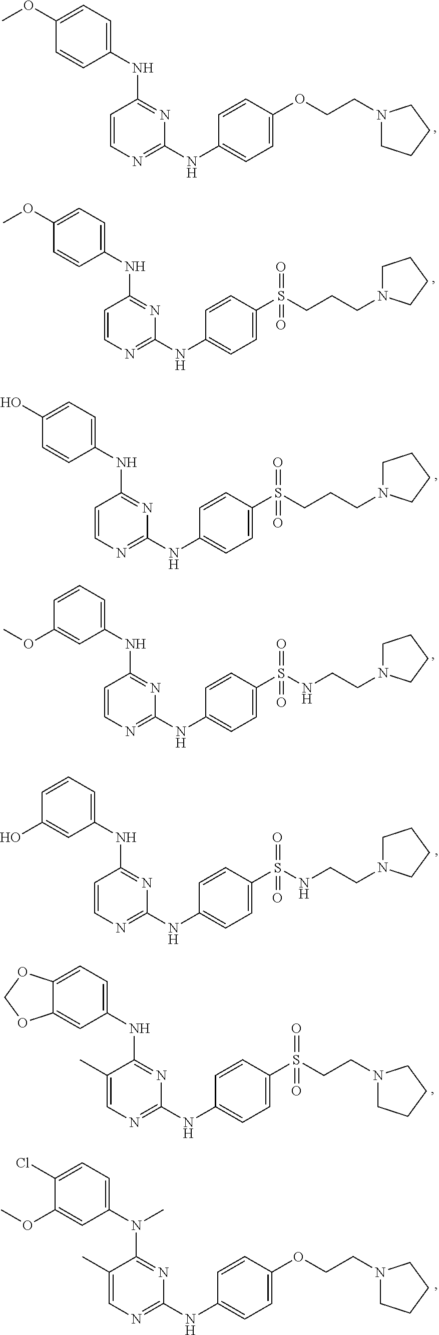

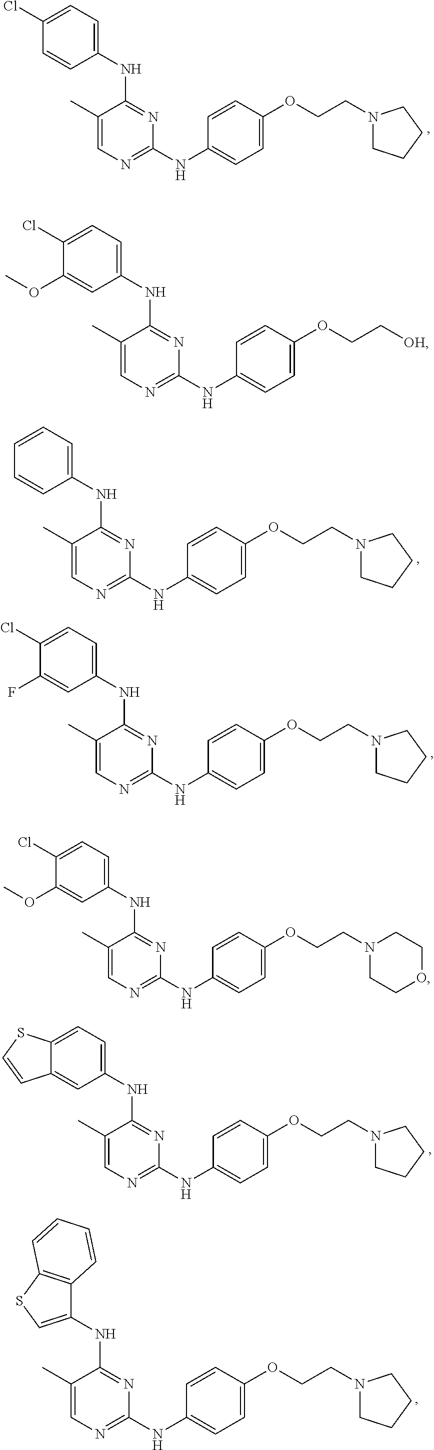

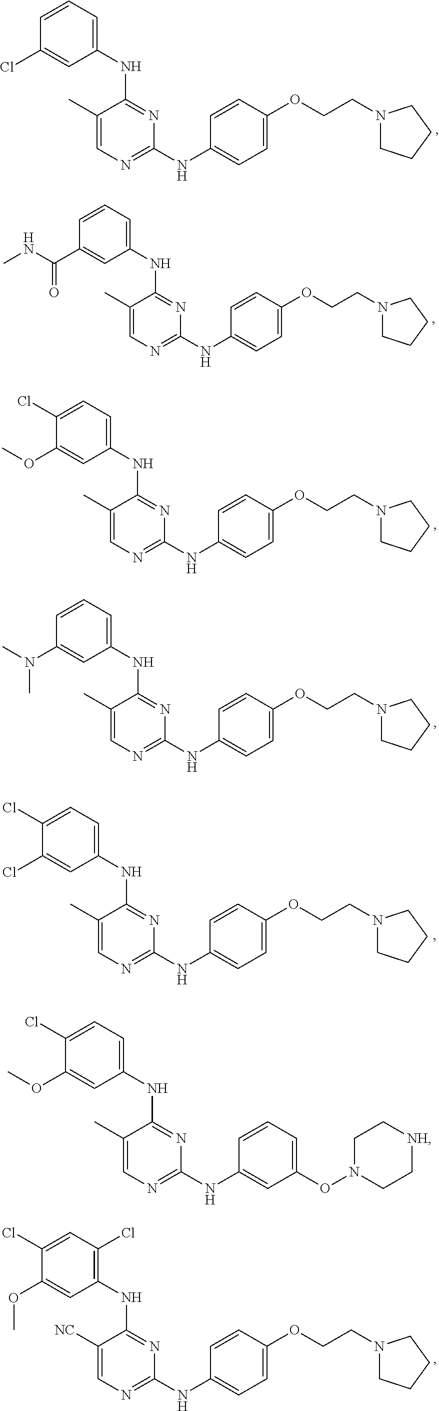

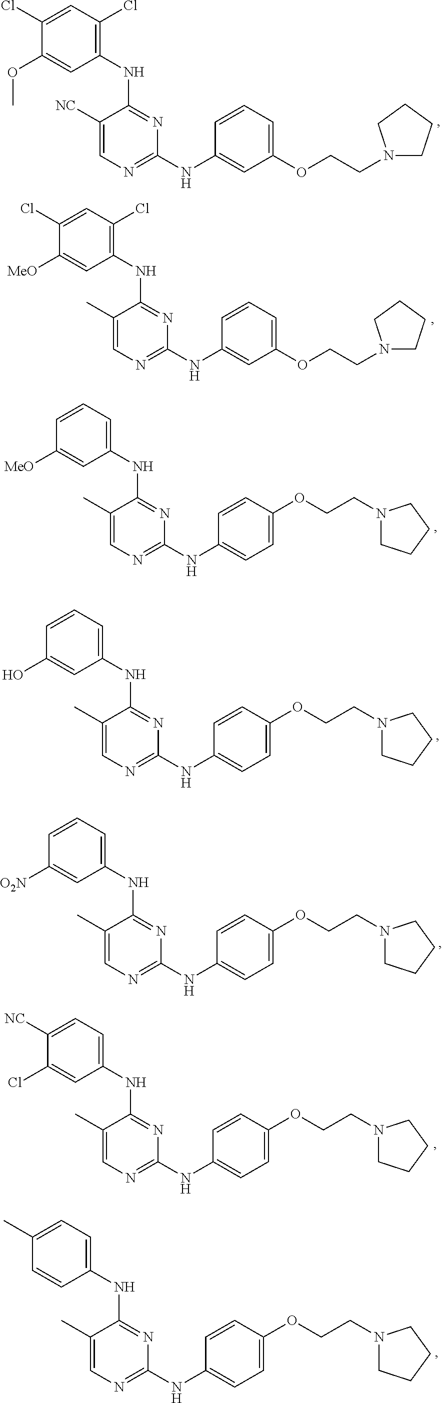

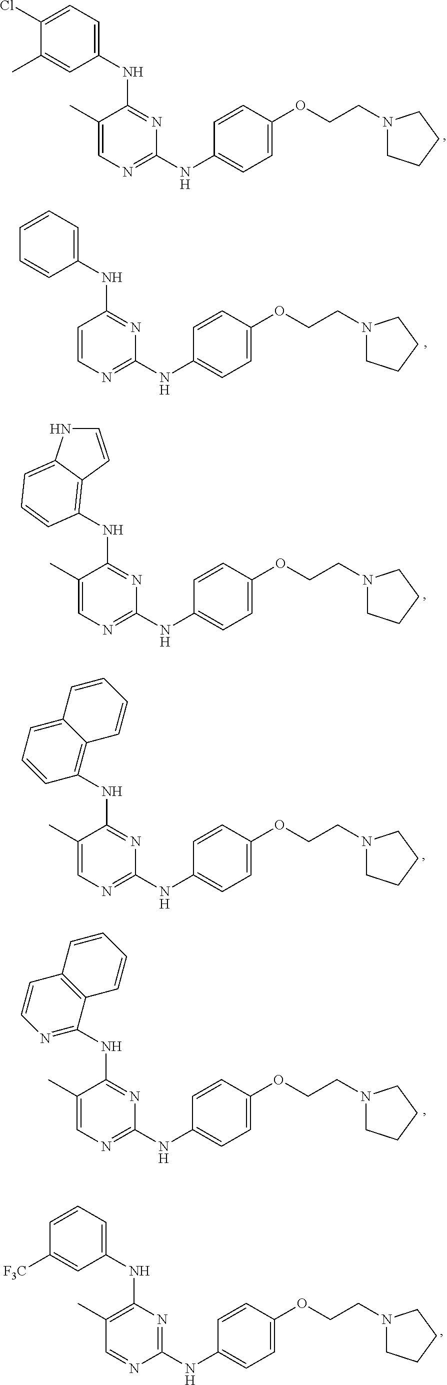

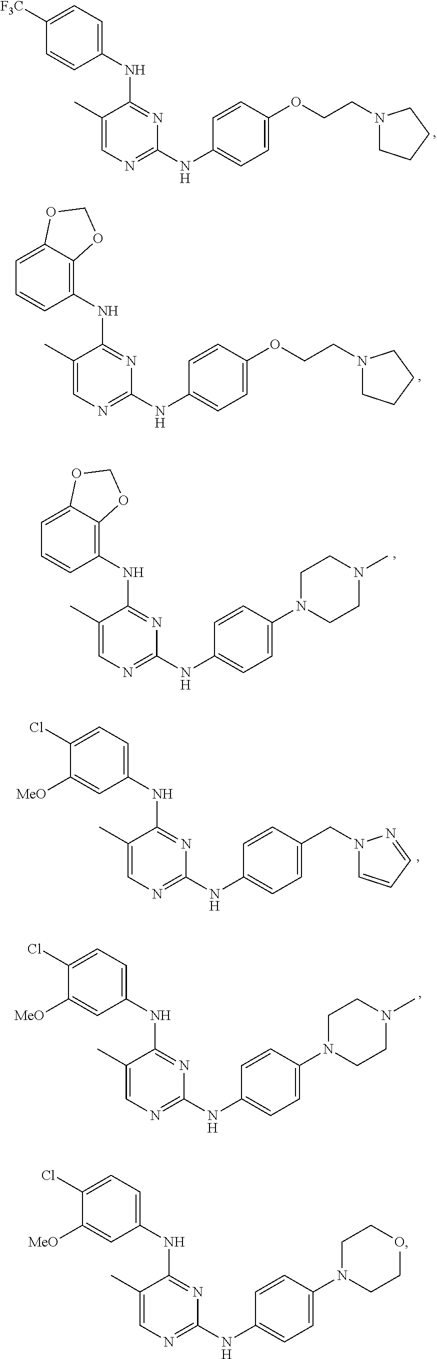

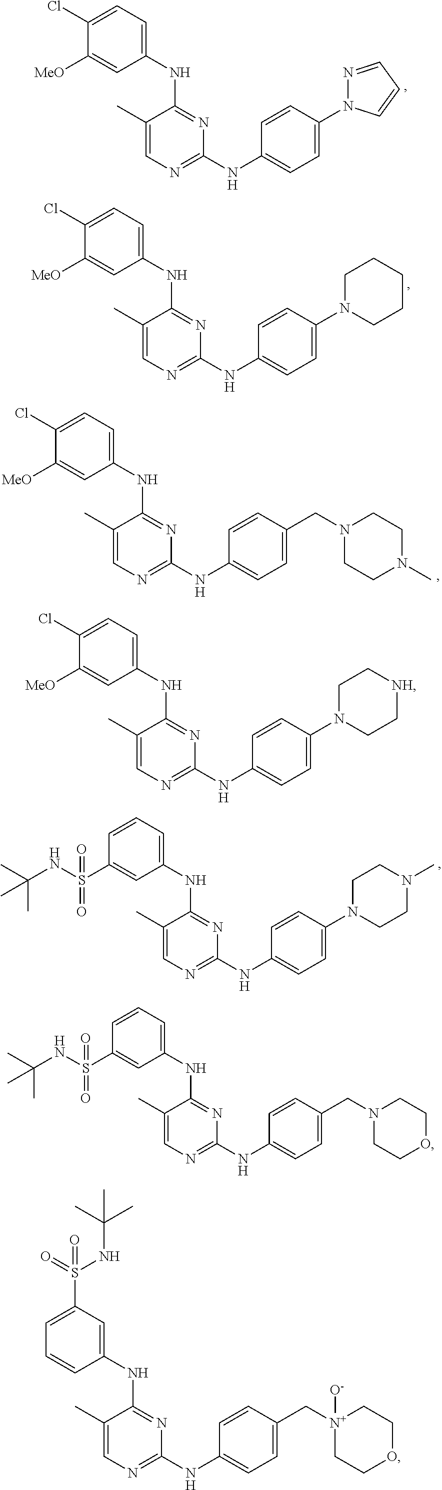

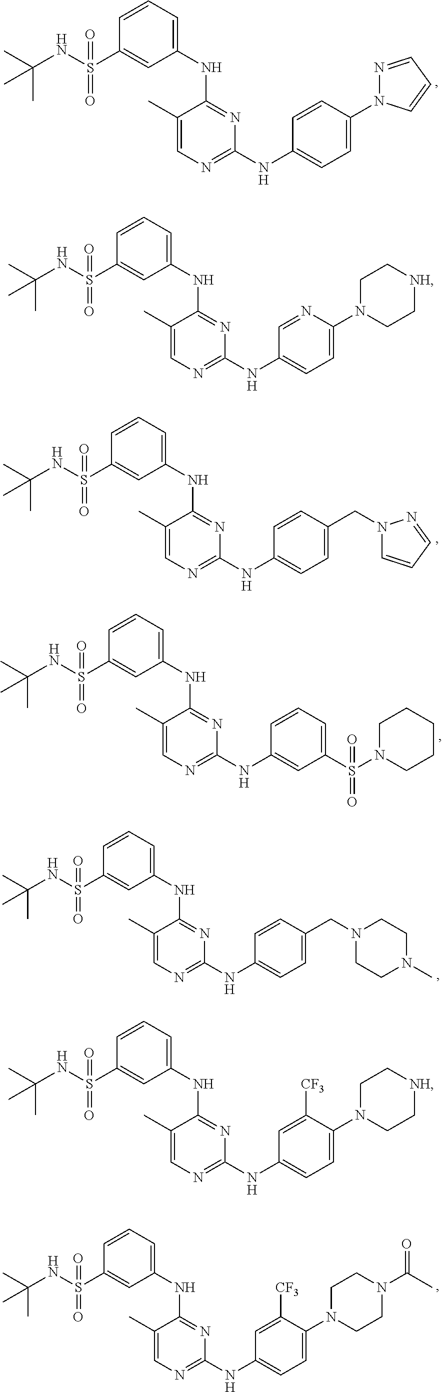

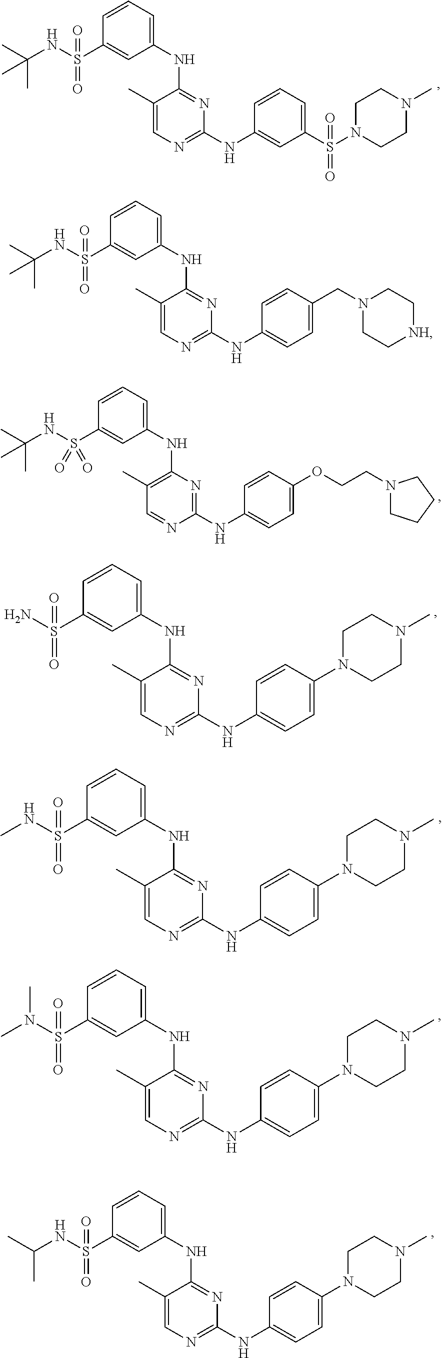

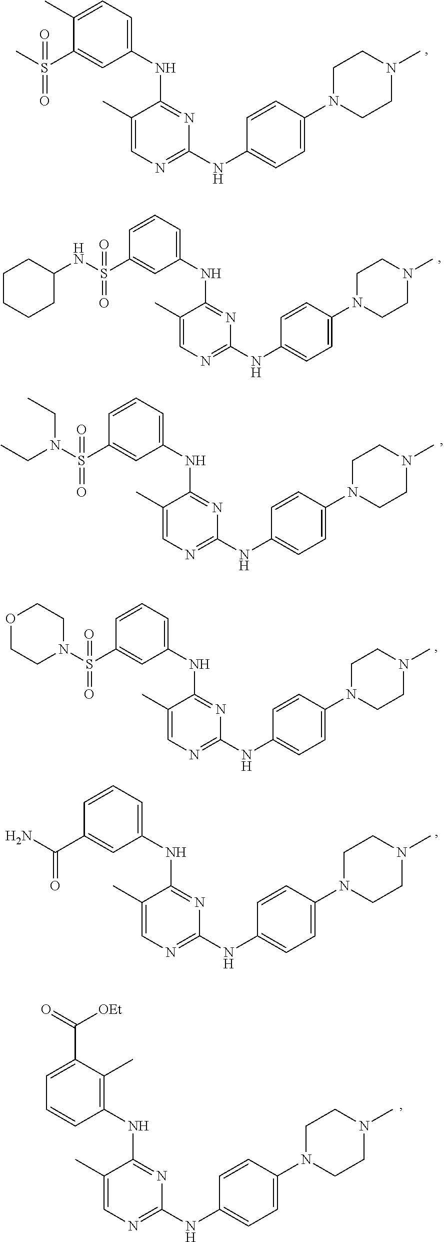

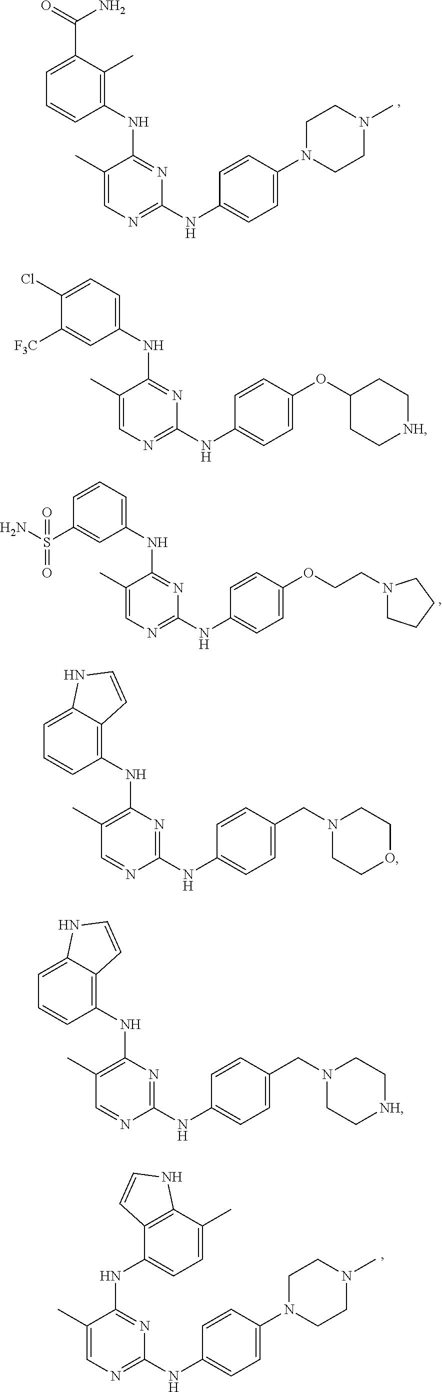

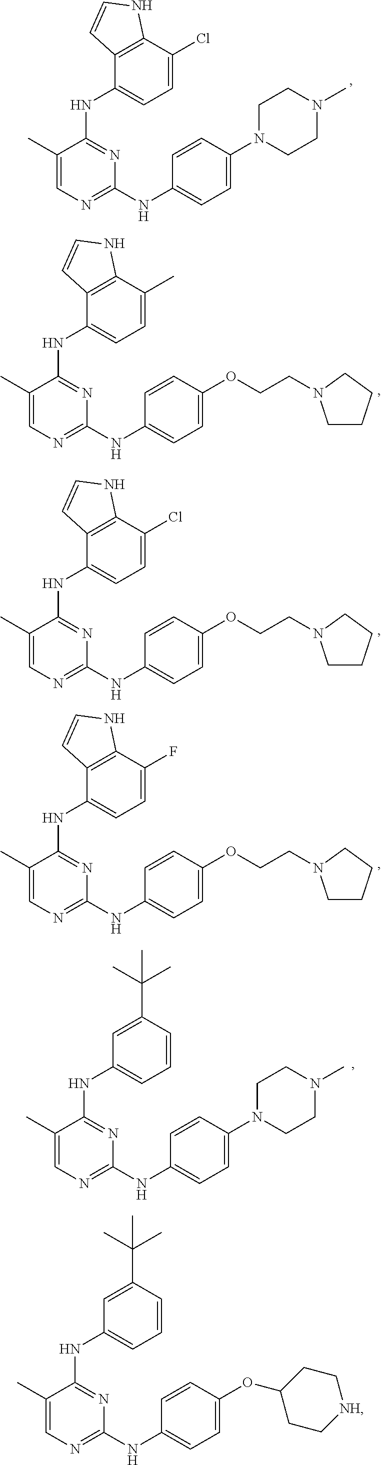

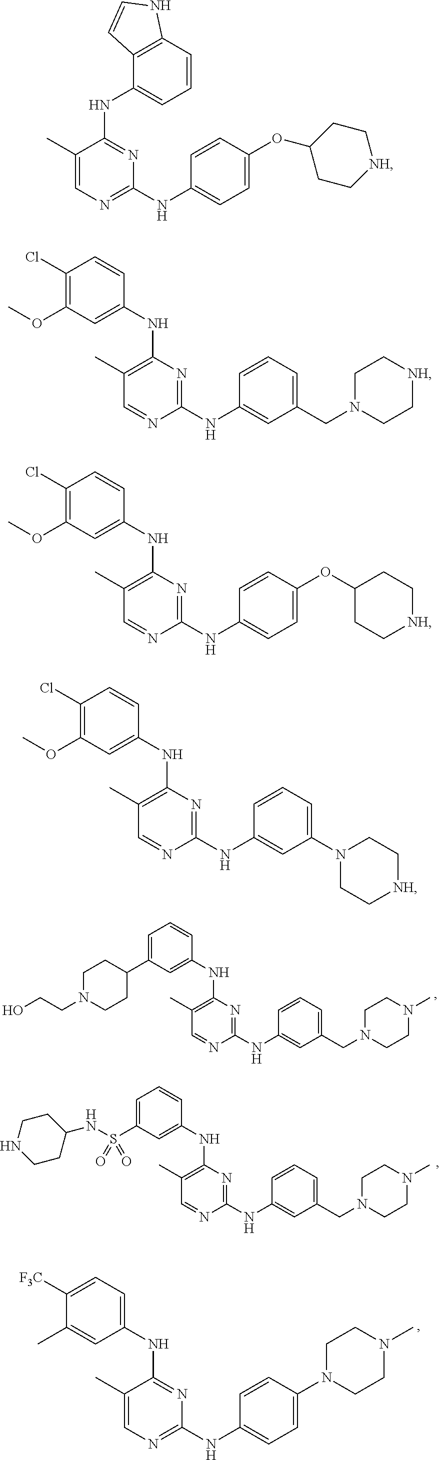

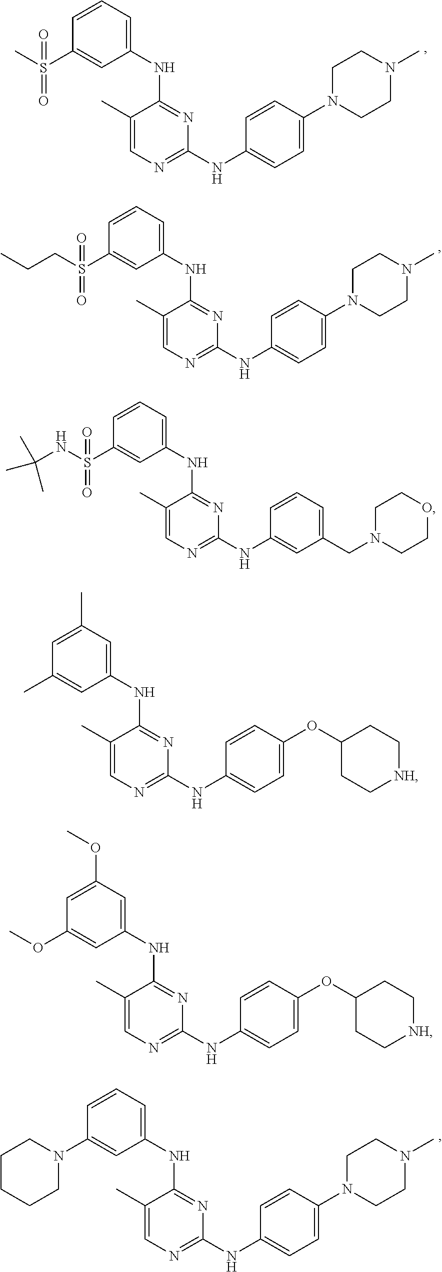

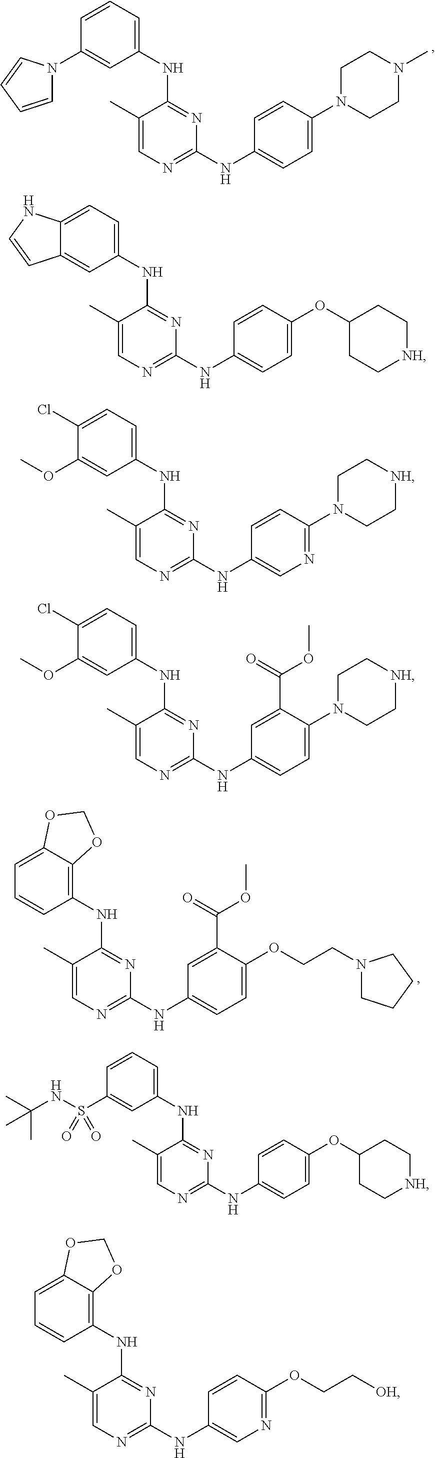

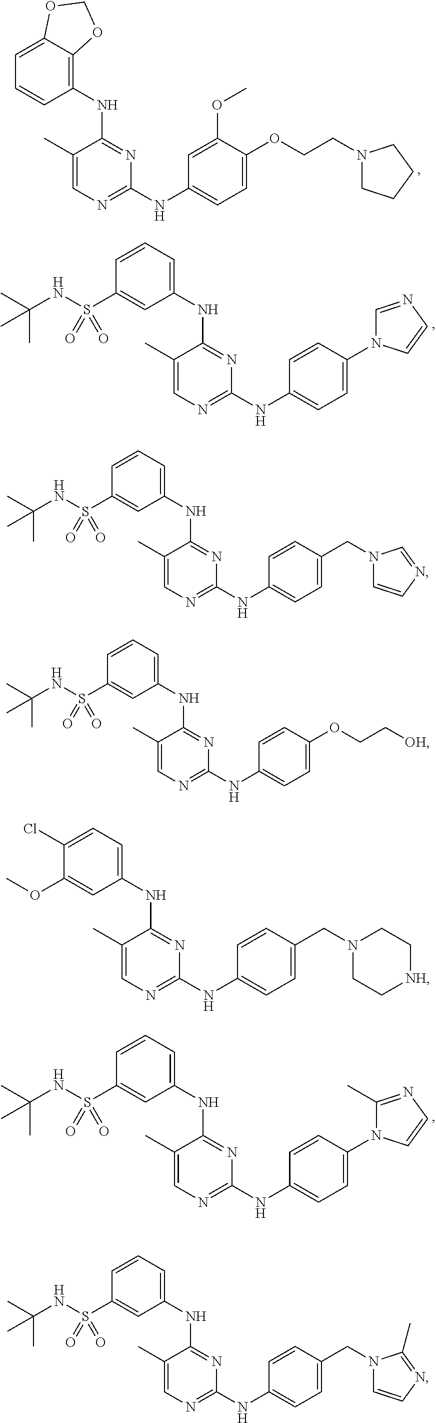

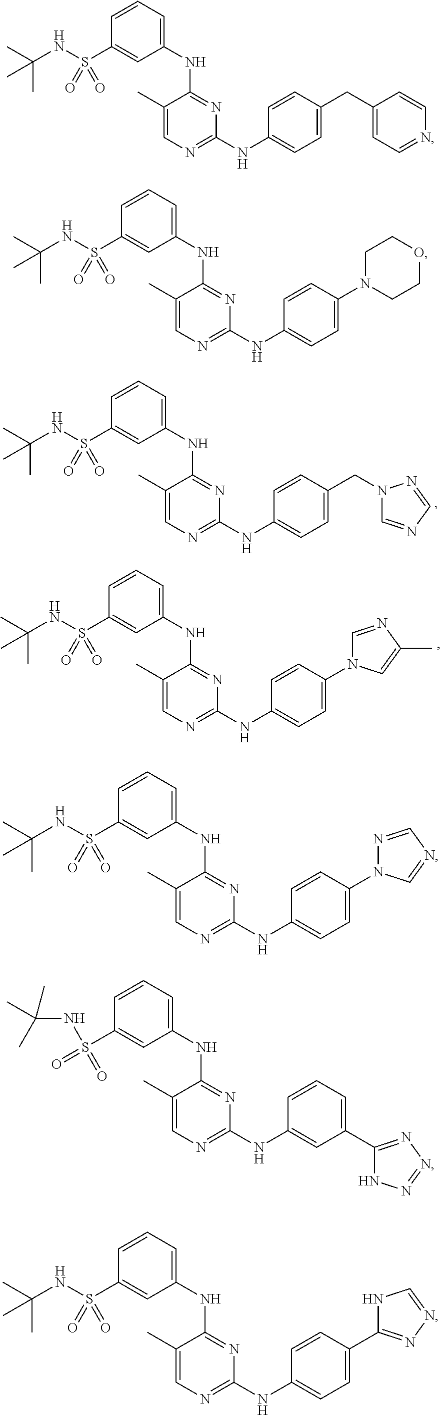

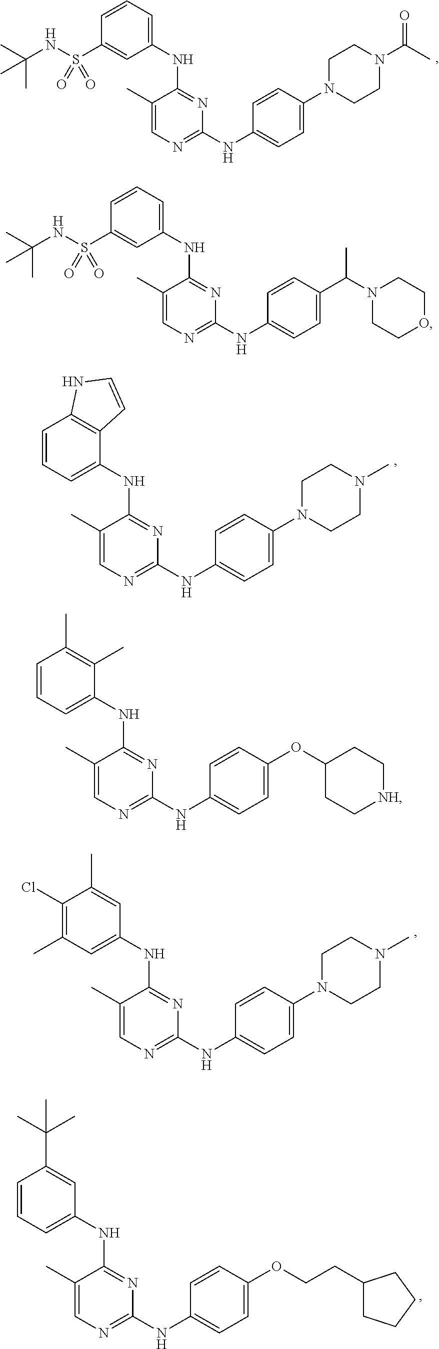

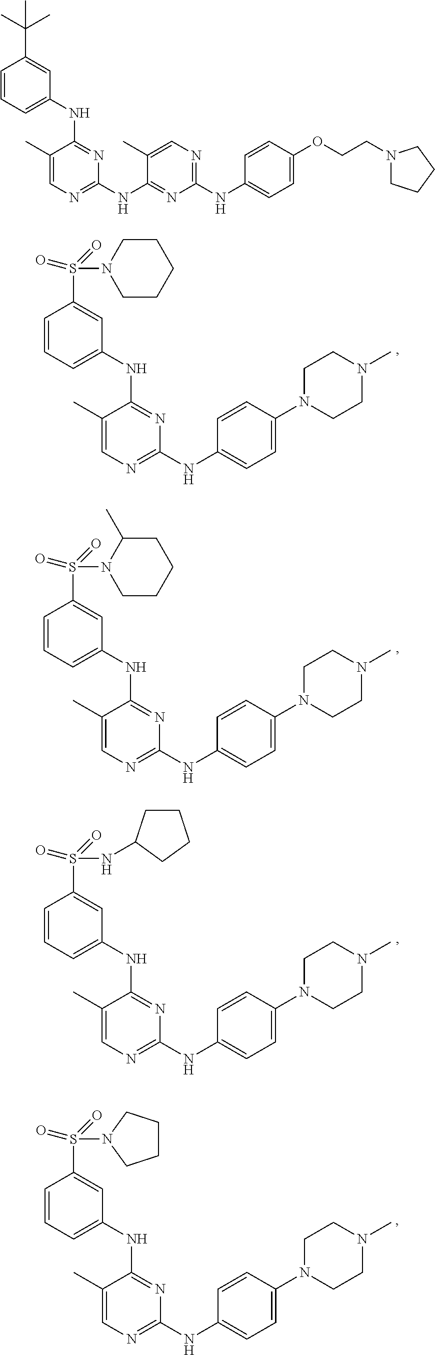

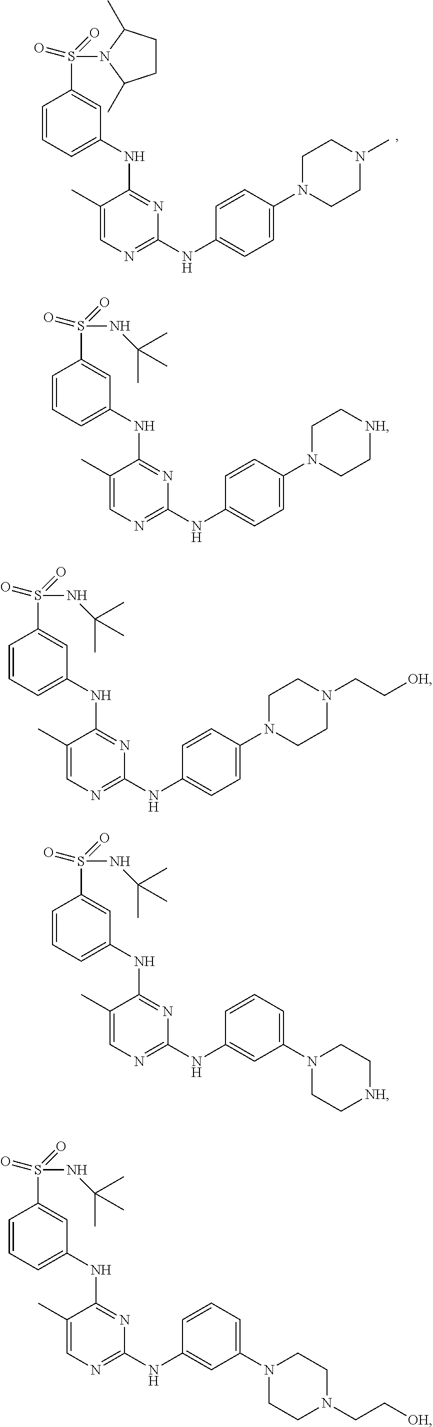

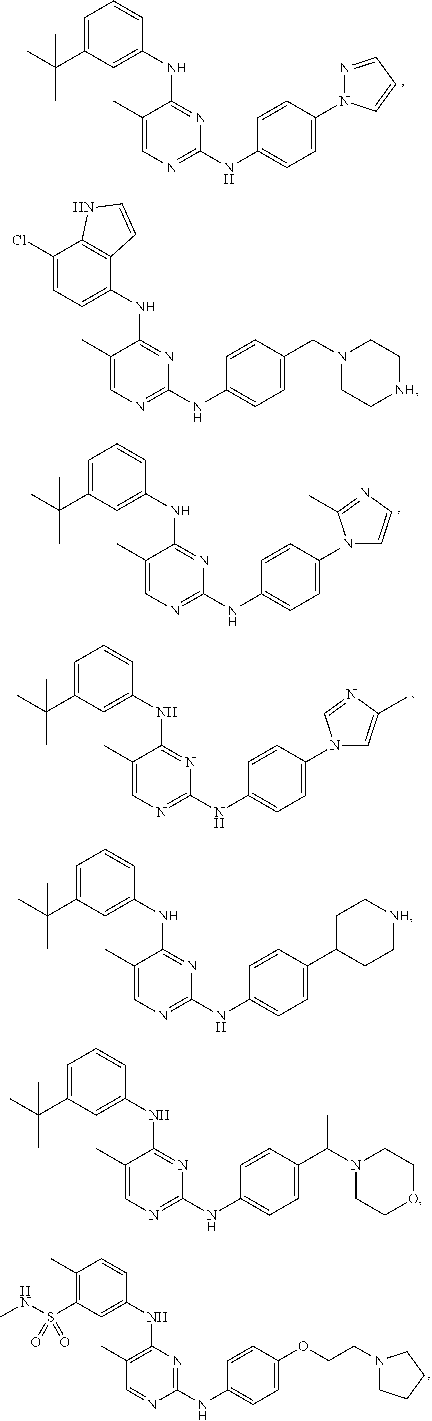

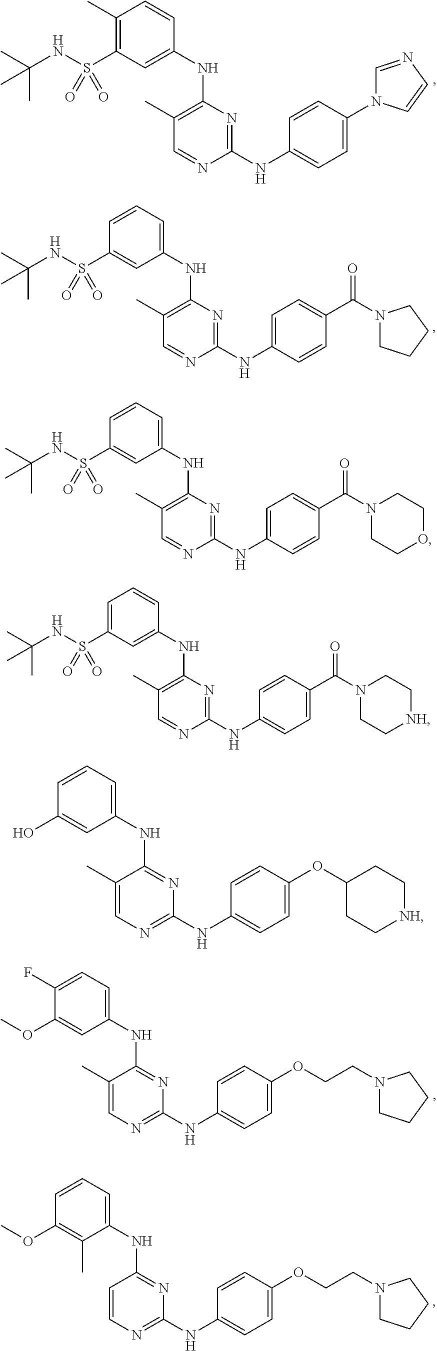

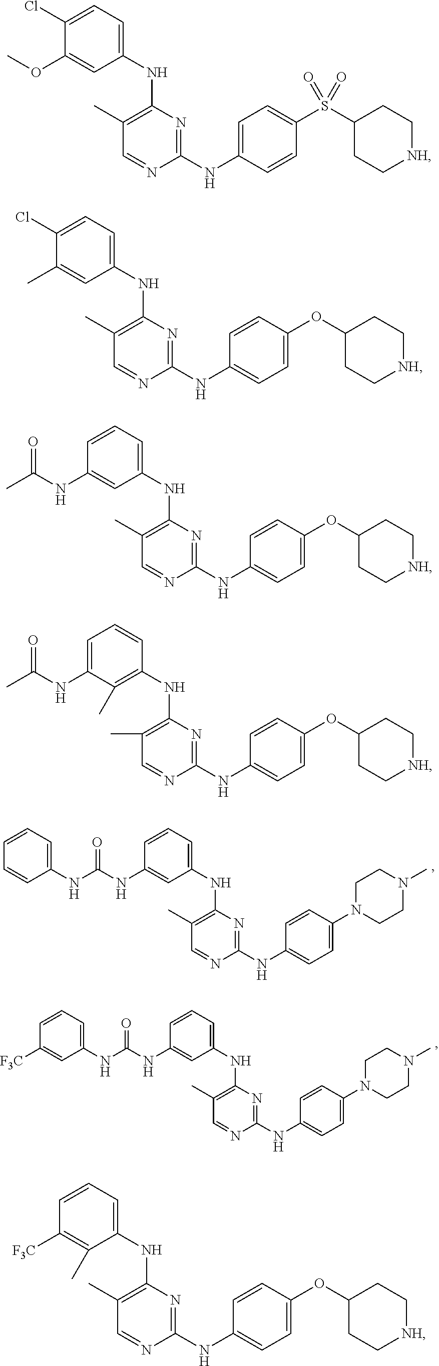

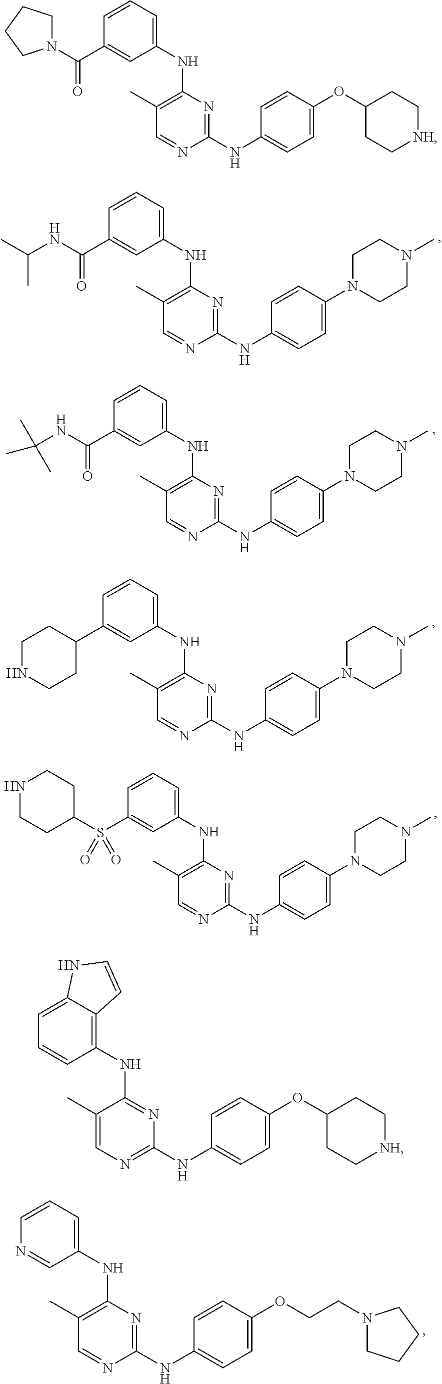

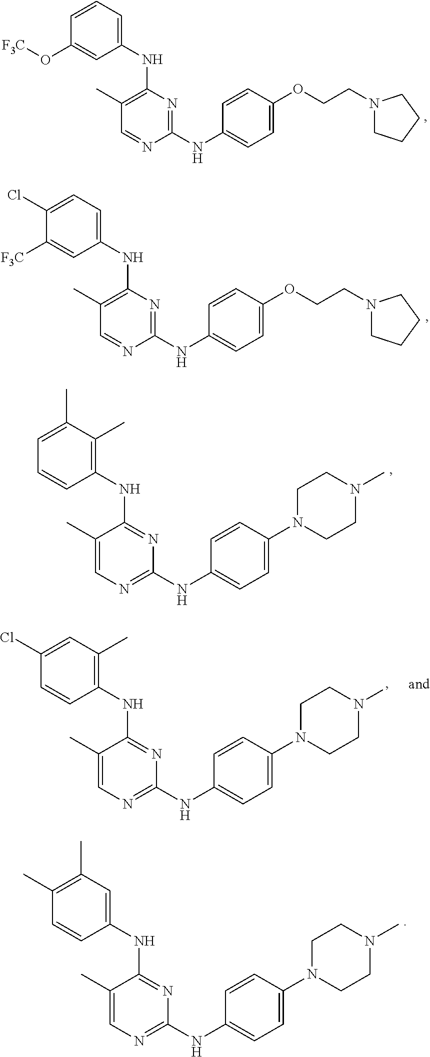

14. A method of treating thalassemia, or ameliorating or delaying at least one symptom of thalassemia in a patient in need thereof, comprising administering a therapeutically effective amount of a compound represented by a first moiety chemically connected to a second moiety, or a pharmaceutically acceptable salt or N-oxide thereof, wherein the first moiety is selected from the group consisting of: 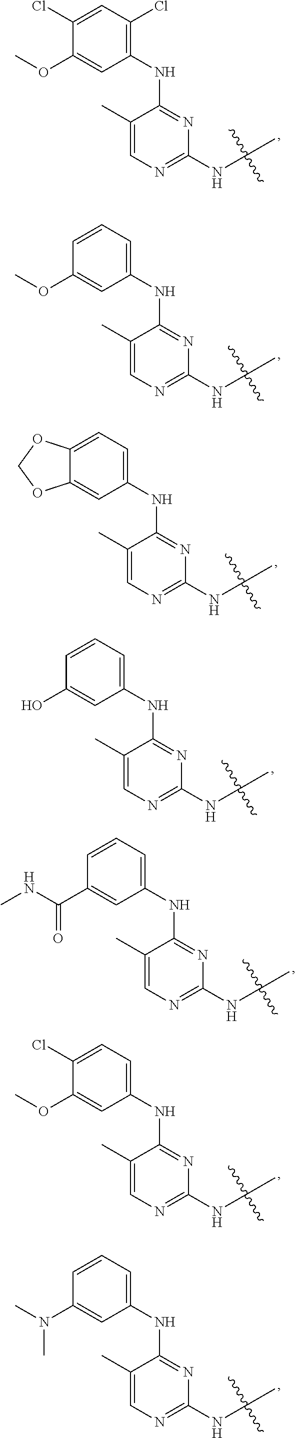 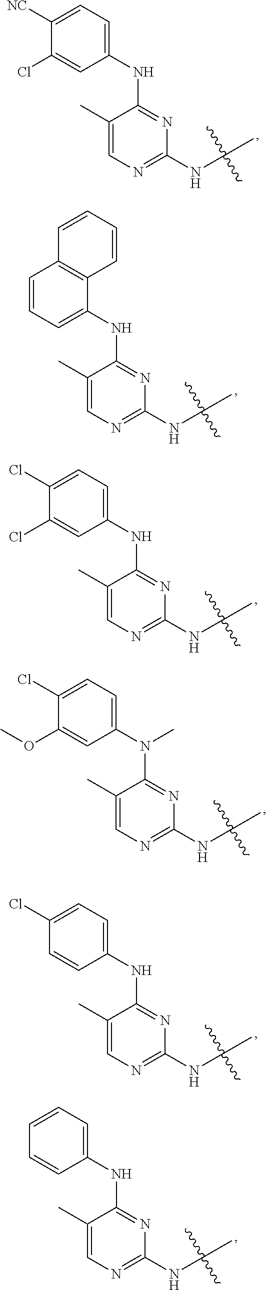 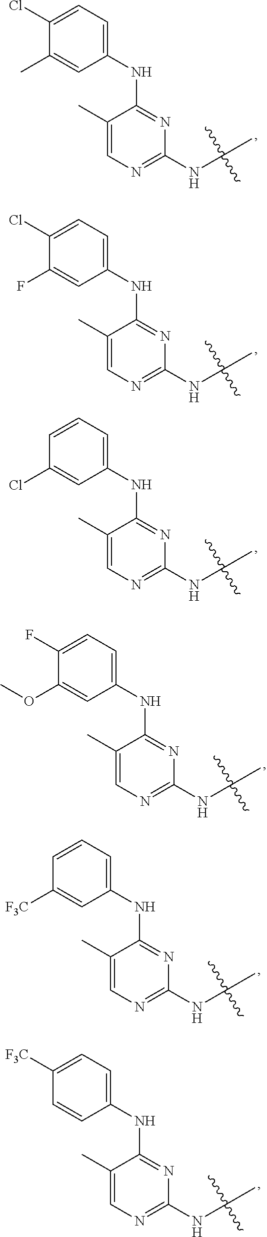 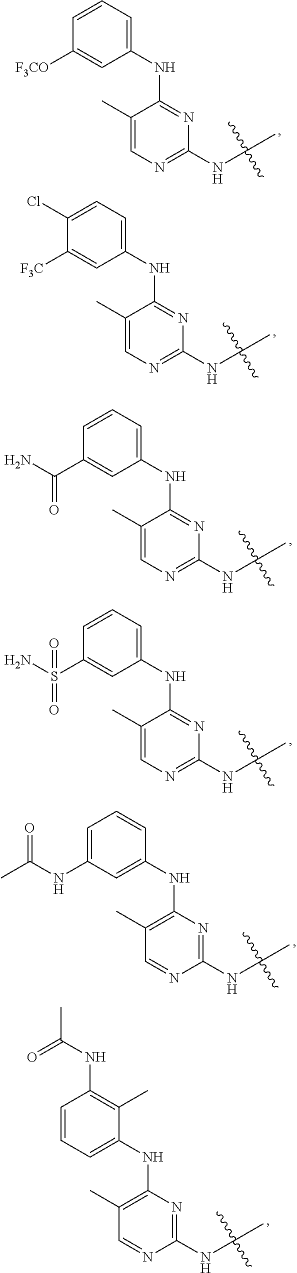 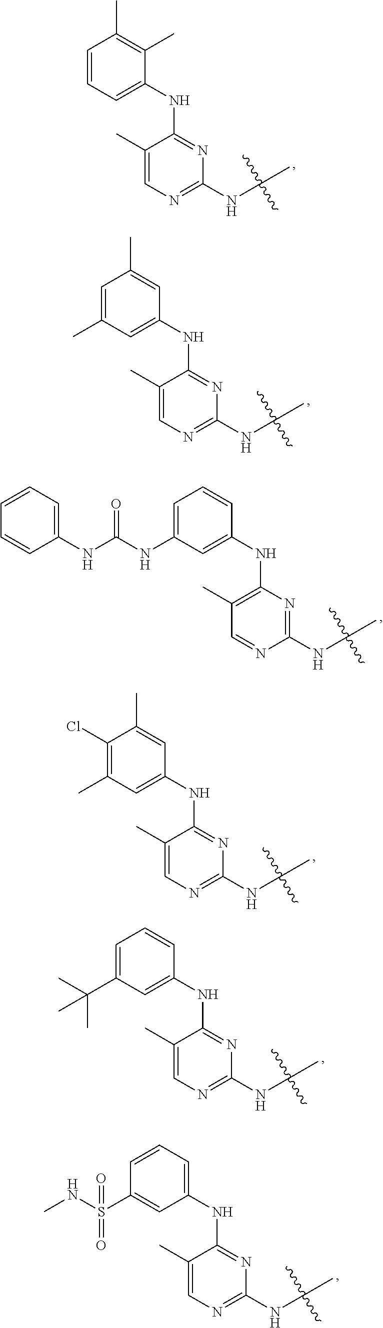 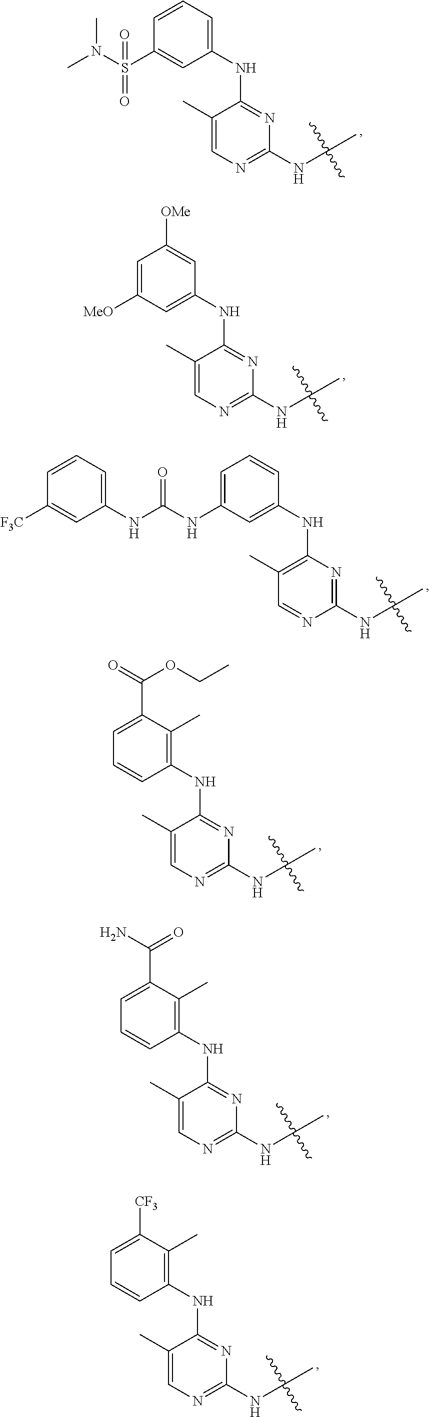 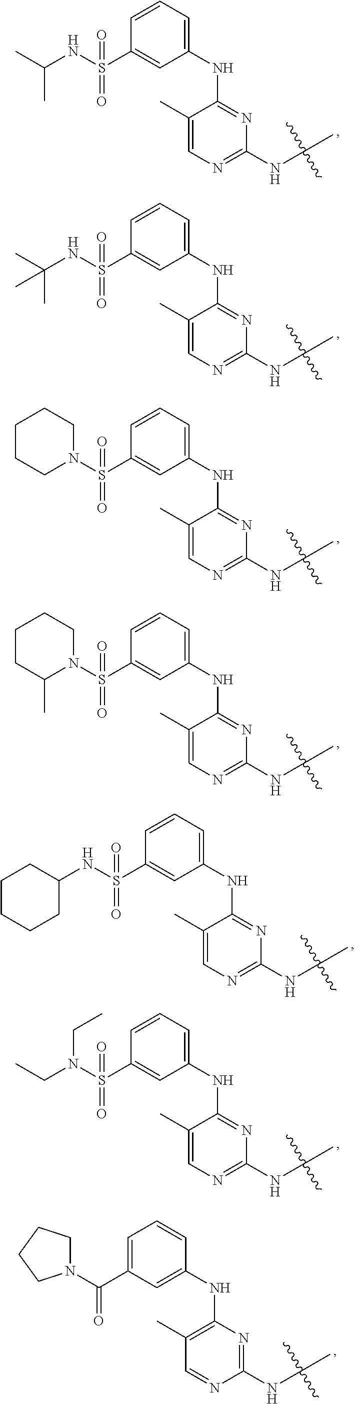 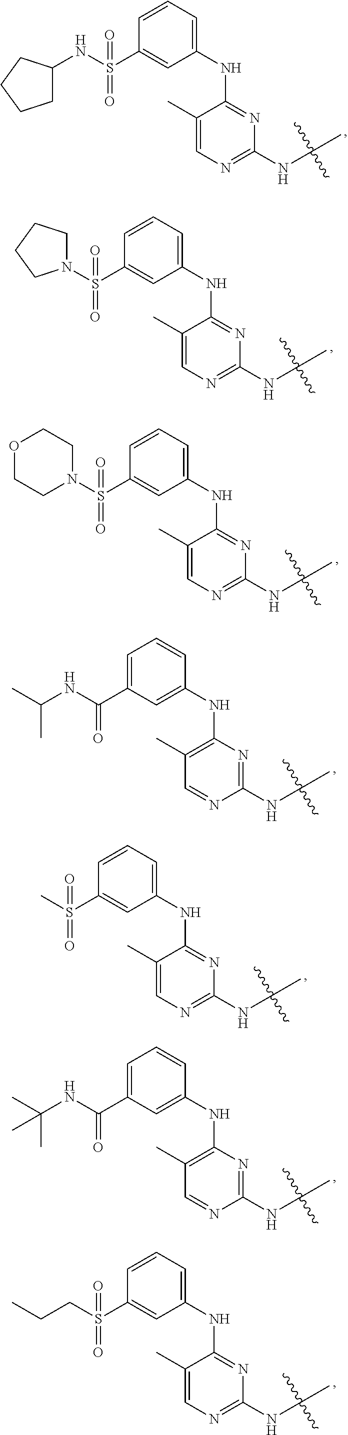 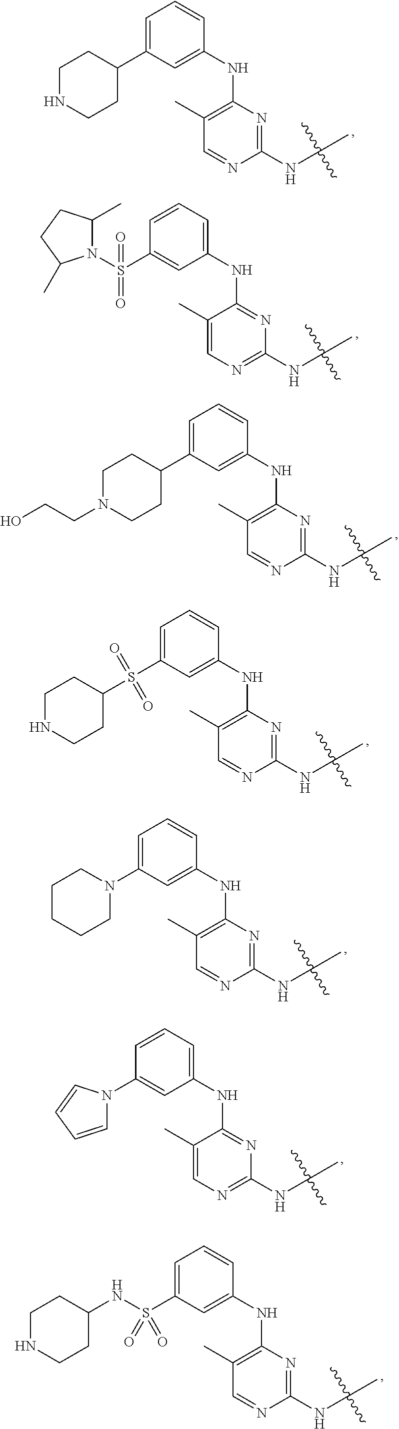 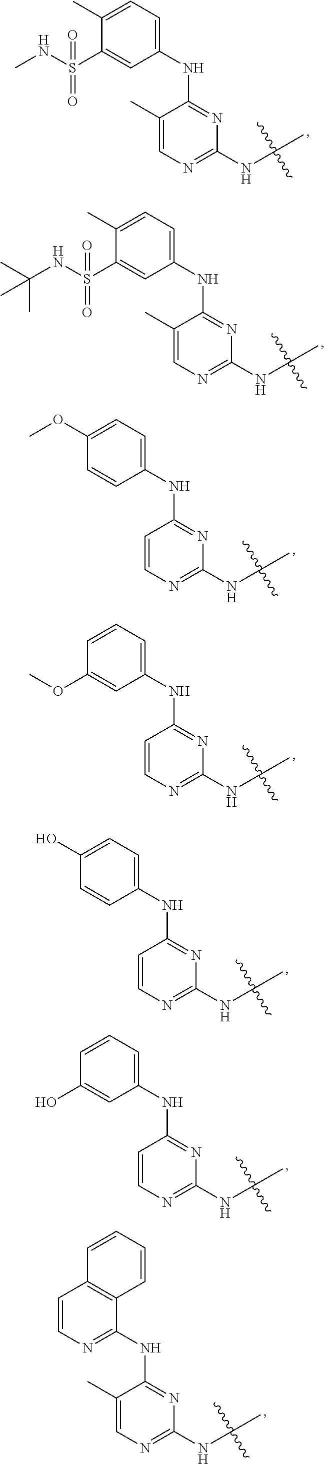 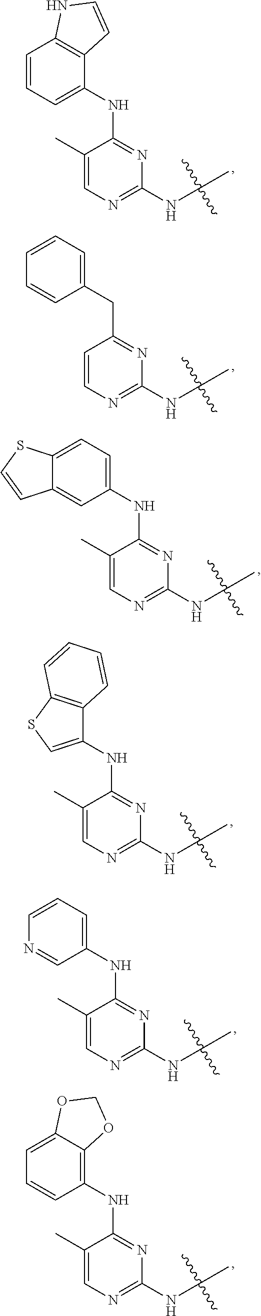 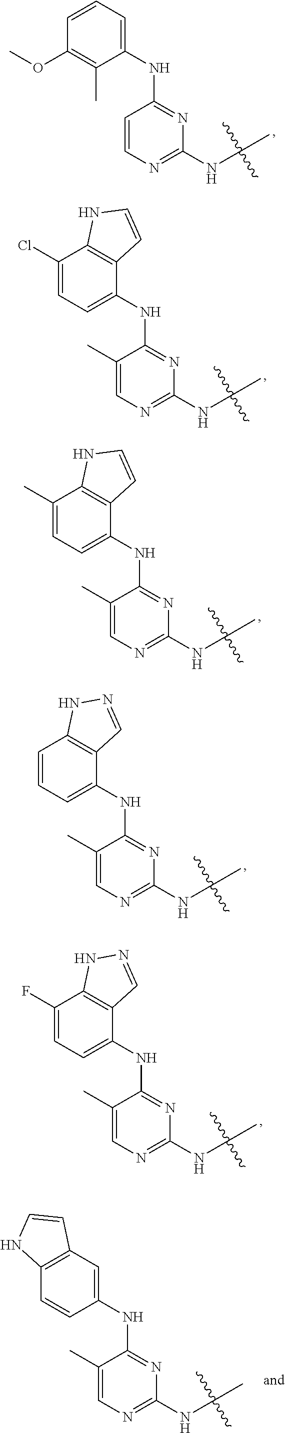
wherein the second moiety is selected from the group consisting of:

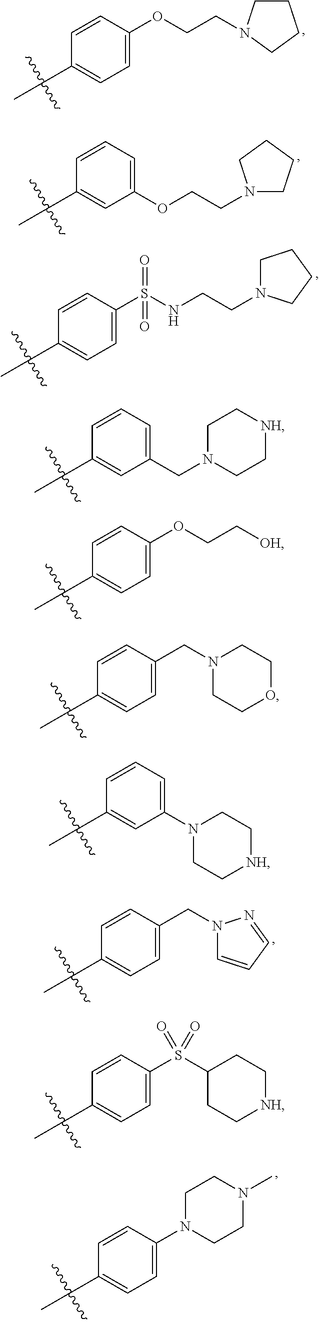

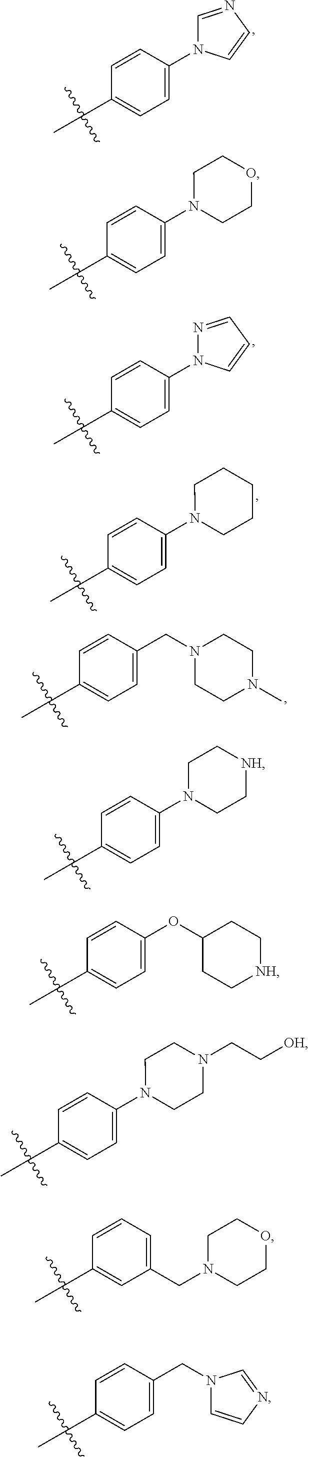

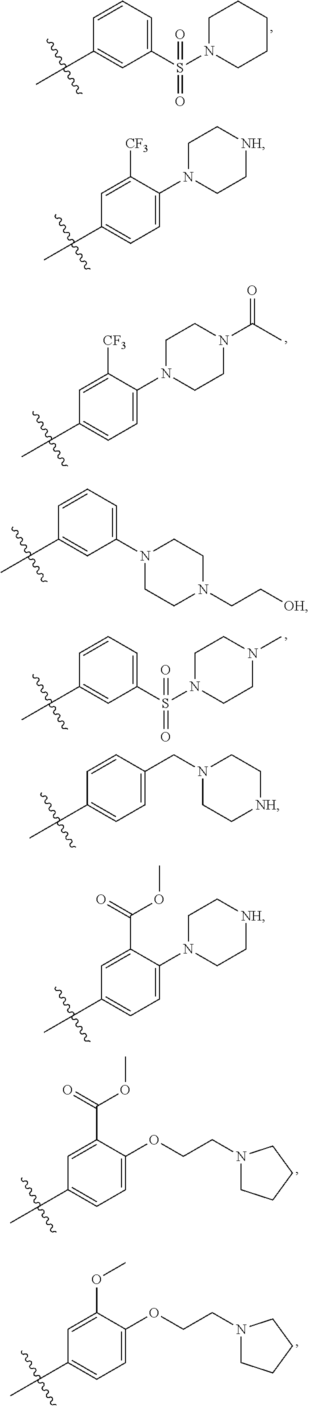

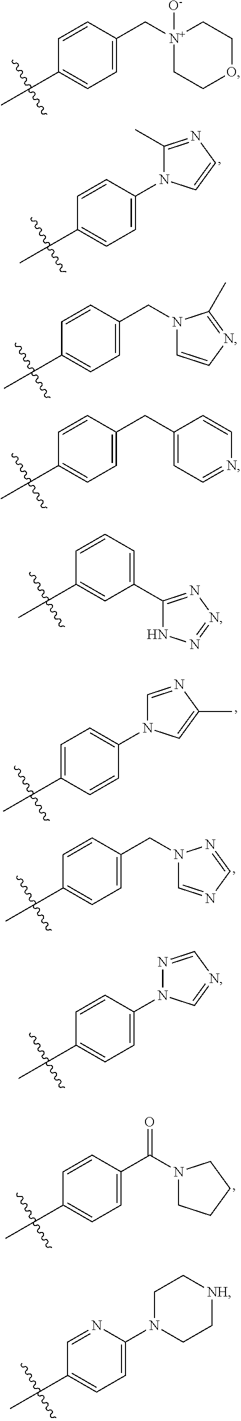

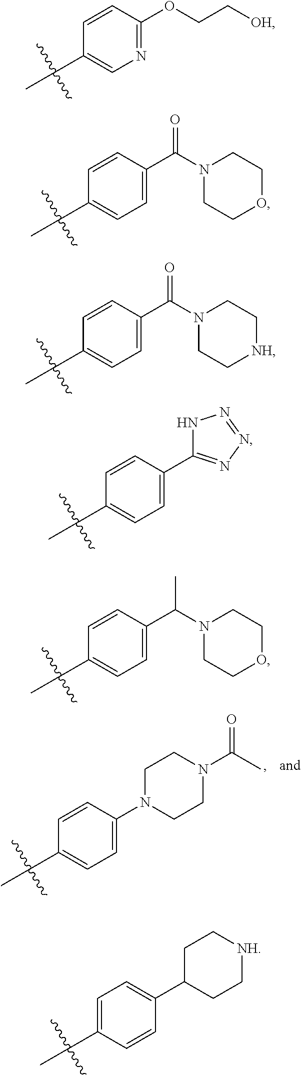

15. The method of claim 14, wherein the symptom is selected from the group consisting of iron overload and an enlarged spleen.

16. The method of claim 1, wherein the symptom is selected from the group consisting of iron overload and an enlarged spleen.

17. A method of treating thalassemia comprising administering to a patient in need thereof a therapeutically effective amount of a compound selected from the group consisting of: 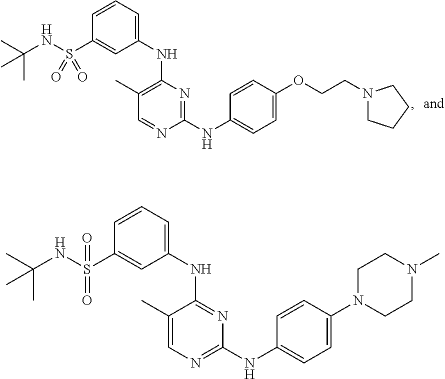
or a pharmaceutically acceptable salt thereof.

18. The method of claim 1, wherein the thalassemia is alpha-thalassemia.

19. The method of claim 1, wherein the thalassemia is beta-thalassemia.

20. The method of claim 17, wherein the thalassemia is alpha-thalassemia.

21. The method of claim 17, wherein the thalassemia is beta-thalassemia.
